SEQUENCE LISTING (15 sequences)

SEQ ID NO: 1 - protein, 5 aa, Artificial Sequence
  “Linker”
SGGGS

SEQ ID NO: 2 - DNA, 32 bp, Artificial Sequence
  “Primer”
ccactagtggcgaagagcctcagaatttaatc

SEQ ID NO: 3 - DNA, 33 bp, Artificial Sequence
  “Primer”
gagaattctattataagcctaaggcagcttgac

SEQ ID NO: 4 - DNA, 45 bp, Artificial Sequence
  “Primer”
ctgaaccagttatgtgtgttggctgagaaaacgccagtaagtgac

SEQ ID NO: 5 - DNA, 44 bp, Artificial Sequence
  “Primer”
gtttaatgctgaaacattcaccttcgctgcagatatatgcacac

SEQ ID NO: 6 - DNA, 40 bp, Artificial Sequence
  “Primer”
ctgcacttgttgagctcgtgaaagccaagcccaaggcaac

SEQ ID NO: 7 - protein, 20 aa, Artificial Sequence
  “Cyclic Peptide”
SDWVCEFIKSQWFCNVLASG

SEQ ID NO: 8 - DNA, 1473 bp, Artificial Sequence
  “Fusion Protein”
tctagagccgccaccatggagacagacacactcctgctatgggtgctgctgctctgggttccaggttccaccggtgacattgtgctgacccaatctccagcttctttggctgtgtctcttgggcagagggccactatgtcctgcagagccggtgaaagtgttgatatttttggcgttgggtttttgcactggtaccagcagaaaccaggacagccacccaaactcctcatctatcgtgcatccaacctagaatctgggatccctgtcaggttcagtggcactgggtctaggacagacttcaccctcatcattgatcctgtggaggctgatgatgttgccacctattactgtcagcaaactaatgaggatccgtacacgttcggaggggggaccaagctggaaataaaaggtggaggcagtggaggcggtggagaggttcagctgcagcagtccggggcagagcttgtggagccaggggcctcagtcaagttgtcctgcacagcttctggcttcaacattaaagacacctatatgcactgggtgaagcagaggcctgaacagggcctggaatggattggaaggattgatcctgcgaatggtaatagtaaatatgtcccgaagttccagggcaaggccactataacagcagacacatcctccaacacagcctacctgcagctcaccagcctgacatctgaggacactgccgtctattattgtgctccgtttggttactacgtgtctgactatgctatggcctactggggtcaaggaacctcagtcaccgtctcgagcggctccactagtggctctggcaagccaggctctggcgagggctccactagtggcgaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataatagaattca

SEQ ID NO: 9 - protein, 487 aa, Artificial Sequence
  Synthetic Construct
SRAATMETDTLLLWVLLLWVPGSTGDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGGGSGGGGEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSGSTSGSGKPGSGEGSTSGEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 10 - DNA, 750 bp, Artificial Sequence
  “Fusion Protein”
tctagagccgccaccatggagacagacacactcctgctatgggtgctgctgctctgggttccaggttccaccggtgaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagcggccgcagaacaaaaactcatctcagaagaggatctgaatggggccgcatgatagaattcg

SEQ ID NO: 11 - protein, 246 aa, Artificial Sequence
  Synthetic Construct
SRAATMETDTLLLWVLLLWVPGSTGEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAAEQKLISEEDLNGAA

SEQ ID NO: 12 - DNA, 468 bp, Artificial Sequence
  “Fusion Protein”
tctagagccgccaccatggagacagacacactcctgctatgggtgctgctgctctgggttccaggttccaccggtgaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggcggccgcagaacaaaaactcatctcagaagaggatctgaatggggccgcatgatagaattcg

SEQ ID NO: 13 - protein, 152 aa, Artificial Sequence
  Synthetic Construct
SRAATMETDTLLLWVLLLWVPGSTGEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALAAAEQKLISEEDLNGAA

SEQ ID NO: 14 - DNA, 420 bp, Artificial Sequence
  “Fusion Protein”
tctagagccgccaccatggagacagacacactcctgctatgggtgctgctgctctgggttccaggttccaccggtgaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagcggccgcagaacaaaaactcatctcagaagaggatctgaatggggccgcatgatagaattcg

SEQ ID NO: 15 - protein, 136 aa, Artificial Sequence
  Synthetic Construct
SRAATMETDTLLLWVLLLWVPGSTGEVDETYVPKEFNAETFTFHADICTLSEKERQIKKQTALVELVKHKPKATKEQLKAVMDDFAAFVEKCCKADDKETCFAEEGKKLVAASQAALGLAAAEQKLISEEDLNGAA

(19) United States
(12) Patent Application Publication
(10) Pub. No.: US 2012/0076728 A1
(43) Pub. Date: Mar. 29, 2012

(54) HUMAN PROTEIN SCAFFOLD WITH CONTROLLED SERUM PHARMACOKINETICS

(75) Inventors: Anna M. Wu, Sherman Oaks, CA (US); Vania E. Kenanova, Sherman Oaks, CA (US); Tove Olafsen, Reseda, CA (US)

(73) Assignee: The Regents of the University of California, Oakland, CA (US)

(21) Appl. No.: 13/263,069

(22) PCT Filed: Apr. 7, 2010
(86) PCT No.: PCT/US2010/030291

§ 371 Date: Nov. 23, 2011

Related U.S. Application Data
(60) Provisional application No. 61/167,844, filed on Apr. 8, 2009.

(51) Int. Cl. A61K 38/38; A61K 49/00; A61P 35/00; C12N 9/96; C07K 19/00; A61K 51/08; B82Y 5/00; B82Y 15/00

(52) U.S. Cl. 424/1.69; 530/363; 424/9.1; 514/19.3; 435/188; 977/774; 977/773; 977/795; 977/927

(57) ABSTRACT

This invention provides constructs comprising a protein scaffold, wherein the scaffold comprises Domain III, Domain IIIa, or Domain IIIb of human serum albumin or a polypeptide having substantial sequence identity to the Domain III, the Domain IIIa, or the Domain IIIb; and a targeting moiety in covalent linkage to the protein scaffold; and a therapeutic moiety and/or an imaging moiety in covalent linkage to the protein scaffold. The scaffold can be modified to tune the serum pharmacokinetics of the construct. In addition to methods of making the constructs, therapeutic, imaging and diagnostic uses of the constructs are also provided.

### CROSS-REFERENCES TO RELATED APPLICATIONS

This application claims priority benefit of U.S. Provisional Application Ser. No. 61/167,844, filed Apr. 8, 2009, the contents of which are incorporated herein in their entirety for all purposes.

### STATEMENT AS TO RIGHTS TO INVENTIONS MADE UNDER FEDERALLY SPONSORED RESEARCH AND DEVELOPMENT

This invention was made with Government support of Grant No. Number CA086306, awarded by the National Institutes of Health. The Government has certain rights in this invention.

### REFERENCE TO SEQUENCE LISTING SUBMITTED ELECTRONICALLY

The sequence listing contained in the file named “008074-5027_seqlist.txt”, created on Nov. 21, 2011 and having a size of 25.9 kilobytes, has been submitted electronically herewith via EFS-Web, and the contents of the txt file are hereby incorporated by reference in their entirety.

### FIELD OF THE INVENTION

This invention relates to constructs, their compositions, and their uses, in which the constructs comprise human serum albumin Domain III as a scaffold to which one or more targeting moieties and one or more an imaging, diagnostic, or therapeutic moieties are attached.

### BACKGROUND OF THE INVENTION

Within the past decade, combinatorial library technology has yielded a large number of molecules, including peptides, aptamers and small chemical molecules, selected to bind various targets or tissues with great specificity and affinity (Aina et al., 2007; Barbas and White, 2009; Bembenek et al., 2009). Yet, when administered in vivo, these molecules often exhibit suboptimal pharmacokinetics (PK) characterized by transient serum persistence and inability to accumulate at the target site to sufficient levels for either imaging or therapy applications. To address this problem, such targeting molecules can be attached to a scaffold. There are several scaffolds described in recent reviews (Gronwall and Stahl, 2009; Nuttall and Walsh, 2008). However, they are either of non-human origin (e.g. affibody, derived from Staphylococcal Protein A (Friedman et al., 2007), camelid and shark single domain antibody isotypes (Saerens et al., 2008), cysteine knot miniproteins derived from plant cyclotides (Simonsen et al., 2008)) or are not capable of providing controllable PK (ankyrins, adnectins, avimers, lipocalins and anticalins (Nuttall and Walsh, 2008)).

Human serum albumin (HSA; 67 kDa) is the most abundant protein in the human body (30-50 g/L) and has already been incorporated into an approved pharmaceutical (i.e. Albuferon®, Novartis). In preclinical studies, HSA has successfully been utilized as a carrier molecule for drug delivery (Burger et al., 2001; Kratz et al., 2000; Wosikowski et al., 2003) and a vector for gene delivery (Aina et al., 2007). As a fusion protein, HSA has demonstrated its ability to improve the PK of molecules, such as interferon-α (Osborn et al., 2002), interleukin-2 (Melder et al., 2005), recombinant bispecific antibody molecule (Muller et al., 2007) or scFv antibody fragment (Yazaki et al., 2008). Similar to IgG, HSA interacts with the neonatal Fc receptor—FcRn, also known as Brambell receptor (Chaudhury et al., 2003). This interaction is responsible for the extended serum persistence of albumin. Briefly, albumin molecules are taken in the endosomes of vascular endothelial cells by fluid phase pinocytosis from the circulation. In the early endosome (˜pH 6.5), albumin binds the FcRn, which resides within this compartment. Upon fusion of the endosome with a lysosome, the unbound content of the endosome is released for degradation, while FcRn-bound albumin is protected. The endosome cycles back to the apical side of the endothelial cell, facing the neutral environment (pH 7.4) of the circulation, where albumin is released by the FcRn back into blood. Specifically, HSA domain III (DIII; 23 kDa) has been shown to bind FcRn in a pH dependent manner (Chaudhury et al., 2006). Three conserved histidine residues (H535, H510 and H464) in HSA DIII have been hypothesized to play a role in the HSA-FcRn interaction (Bos et al., 1989; Chaudhury et al., 2006).

Currently, the most successful targeting agents used for cancer therapy in the clinic are intact antibodies (e.g., Trastuzumab, Rituximab, Bevacizumab). The advantage of using antibodies is that in addition to their superb target affinity and specificity, and good safety profile, they also possess the necessary pharmacokinetics (PK) to achieve therapeutic effect. Antibodies owe their prolonged circulation persistence predominantly to their Fc domain interactions with FcRn. In addition to being twice the size of HSA DIII, Fc domains of antibodies interact with additional endogenous Fc receptors. This biological function may lead to unwanted side effects in clinical applications. The disadvantages of antibodies also include certain limitations with target accessibility, but predominantly the lengthy, highly laborious process of production, which also increases antibody drug cost.

Other targeting moieties, including peptides and aptamers can also be selected to exhibit nanomolar affinity and high specificity for various targets, and are much faster and cheaper to make than antibodies. A major drawback is that these low molecular weight targeting agents typically clear very rapidly from the circulation, with typical serum half-lives in the order of minutes. This leads to low target uptake and limits their potential for clinical use in diagnostic imaging and therapy. In modern medicine, the ability to dial in a desirable PK for targeted imaging and therapeutic agents is highly valued. The provided compositions for, and methods of, in vivo treatment and imaging using molecules that can target a biomolecule and have extended serum persistence with a spectrum of circulation half lives without significantly changing molecular mass. The invention addresses the need for low molecular weight, low or non-immunogenic agents that can provide tumor targeting molecules, such as peptides, aptamers or small chemicals, with the appropriate pharmacokinetic properties needed for in vivo applications, including imaging and/or therapy.

### BRIEF SUMMARY OF THE INVENTION

In a first aspect, the invention provides for the use of HSA DIII as a scaffold in making constructs comprising HSA-DIII and one or more small molecule targeting agents conjugated to the HSA-DIII, as well as one or more of an imaging moiety or a therapeutic moiety conjugated to the HSA-DIII. The HSA-DIII scaffold or carrier can be modified to provide constructs having tailored pharmacokinetics (PK) and also provides opportunities for multivalence and/or multiple specificities, and residues for attachment of functional groups.

In some embodiments, the invention provides a construct comprising a) a protein scaffold, wherein the scaffold comprises Domain III, Domain IIIa, or Domain IIIb of human serum albumin or a variant thereof selected for its altered FcRn receptor binding properties; b) a targeting moiety in covalent linkage to the protein scaffold; and c) a therapeutic moiety or an imaging moiety in covalent linkage to the protein scaffold.

In another aspect, the invention provides methods of detecting a biomolecule associated with a disease or condition in a subject by administering to a subject suspected of having, or having, the disease or condition a construct according to the invention, wherein the targeting moiety of the construct binds the biomolecule and the imaging agent bound to the construct is detected. In some embodiments, the presence of absence of the disease or condition is diagnosed.

In still another aspect, the invention provides a method of targeted therapy of a disease or condition associated with the presence of overexpression of a biomolecule in a tissue, said method comprising administering to a subject having the disease or condition a therapeutically effective amount of the construct according to the invention wherein the targeting moiety of the construct binds the biomolecule and the therapeutic agent of the construct treats the disease or condition in the tissue or cell associated with the presence of the biomolecule.

In yet another aspect, the invention contemplates providing a library of modified Domain III proteins having a variety of target specificities predetermined FcRn affinities for use as scaffolds in the design of targeted imaging and therapeutic constructs according to the invention. In another embodiment, the invention provides nucleic acids encoding one or more of the Domain III scaffolds and variants thereof for use according to the invention. In still further embodiments, the invention provides vectors comprising the nucleic acids operably linked to genetic regulatory factors controlling the expression of the Domain III scaffold and also provides cells containing the vectors or nucleic acids.

In all embodiments and aspects of the invention, in some embodiments, there is a proviso that the construct does not comprise either or both Domain I or Domain II of HSA or alternatively that the construct does not comprise a sequence of more than 5, 10, 15, or 20 contiguous amino acids of domain II of HSA.

### BRIEF DESCRIPTION OF THE DRAWINGS

FIG. 1. (A) Gene assembly of the Db-DIII constructs. L-signal peptide leader for mammalian cell secretion; variable light (V_{L}) and variable heavy (V_{H}) antibody chains are joined through an 8 (glycine, serine rich) amino acid linker to form a single chain fragment variable (scFv, 25 kDa). The scFv is connected to the HSA DIII gene by an 18 amino acid linker. DIII is flanked by SpeI and EcoRI restriction sites in a cassette to facilitate the exchange of one DIII with another (e.g. WT for H535A, etc.). (B) Cartoon representation of the Db-DIII protein, where two scFv-DIII molecules form a non-covalent dimer.

FIG. 2. (A) SDS-PAGE of four Db-DIII proteins: H535A, H510A and H464A in lanes 1, 2 and 3, respectively, under NR conditions, and WT in lane 5 under R conditions. (B) Western blot of Db-DIII WT under NR (lane 2; probed with AP-conjugated anti-mouse Fab mAb) and R conditions (lane 3; probed with HRP-Protein L). (C) Size exclusion chromatography, using Superdex 200 column and 0.5 ml/min flow rate. The Db-DIII WT protein eluted at 28.17 min. Purity was estimated by integration of the peak to be about 98%.

FIG. 3. (A) PyMOL model of HSA DIII composed of half domains DIIIa (green) and DIIIb (yellow). Six disulfide bridges are shown in red. The location of residues H535, H510 and H464 is pointed by the arrows. The H464 residue, located in DIIIa was mutated to A to produce the DIII H464A variant Amino acids H535 and H510, located in the DIIIb, were each exchanged with A to produce DIII H535A and DIII H510A variants. (B) Docking model of the HSA DIII (green) and FcRn (orange) and FcRn (orange) molecules. In red are the residues on FcRn that are involved in IgG binding, while residues at the interface of FcRn and DIII molecules interacting with each other are shown in blue. (C) Model of a divalent Db-DIII molecule, where each Db carries two DIII proteins at both C-termini. The scFv monomers composing the Db are in light and dark green, the 18 amino acid linker is in blue and the DIII molecules are in yellow.

FIG. 4. Small animal PET/CT imaging of athymic nude mice xenografted with CEA-positive LS174T (left) and CEA-negative C6 (right) tumors. Mice were injected with ¹²⁴I-labeled Db-DIII proteins (WT, H535A, H510A, or H464A) and the anti-CEA Db as a reference. Mice were imaged for 10 min at 5 different time points with coronal sections shown. Co-registered PET/CT images are included for anatomical reference of the tumor and organ location.

FIG. 5. (A) Tumor-to-soft tissue ROI analysis of the PET images. (B) Blood activity curves generated by quantitation of radioactivity (% ID/g) from the PET images at each time point.

FIG. 6. Cell binding assay. Increasing concentrations of Alexa 647 conjugated HSA, DIII WT, H535A, H510A and H464A proteins were incubated with 293 cells transduced with human FcRn. As a control, Alexa conjugated HSA was incubated with non-transfected 293 cells.

FIG. 7. Blood activity curves of ¹³¹I-labeled HSA and DIII proteins in Balb/c mice.

FIG. 8. DNA and translated protein sequence of Db-DIII. Outlined are specific sequences and starting points of the following DNA and protein segments: restriction enzyme digestion sites, Kozac sequence, leader—a secretion signal peptide, V_{L}, 8 amino acid inter-domain peptide linker, V_{H}, 18 amino acid linker between the Db and HSA DIII; histidine residues H535, H510A and H464 which are individually mutated to alanine for generation of the Db-DIII variants, and two stop codons followed by a restriction enzyme cut site.

FIG. 9. DNA and translated protein sequence of A. DIII WT. Shown are important sequences and starting points of the following DNA and protein segments: restriction enzyme digestion sites used in cloning, Kozac sequence, leader, beginning of HSA DIIIa, HSA DIIIb, histidine residues H535, H510 and H464, c-Myc peptide, two stop codons followed by a restriction enzyme cut site; B. HSA DIIIa; and C. HSA DIIIb.

FIG. 10. Small animal PET/CT imaging of ¹²⁴I-labeled anti-CEA peptide-DIIIb conjugate. Ten minute static scans at 4, 20 and 27 h post injection with coronal sections shown. The CEA positive (LS174T) and CEA negative (C6) tumors are shown with arrows.

### DETAILED DESCRIPTION

The invention provides for the use of HSA DIII as a scaffold in making constructs comprising HSA-DIII and one or more small molecule targeting agents conjugated to the HSA-DIII, and one or more of an imaging moiety or a therapeutic moiety conjugated to the HSA-DIII. By selecting amino acid sequence modifications within domain III which affect binding to the FcRn receptor or provide additional sites of attachment for targeting, therapeutic and imaging moieties, the HSA-DIII scaffold or carrier can be modified to provide constructs having tailored pharmacokinetics (PK) and also provides opportunities for multivalence and/or multiple specificities, and residues for attachment of functional groups.

We have found that low molecular weight tumor targeting molecules are attached, grafted or displayed onto a HSA domain III protein scaffold characterized by intrinsic serum stability, then improved pharmacokinetic profile and target uptake can be achieved. Maximizing tumor accumulation can translate into a stronger signal in imaging applications or a sufficient drug payload delivery in therapy. In addition, the HSA DIII scaffold can provide residues for conjugation of a functional group (e.g. radionuclide, cytotoxic drug, toxin), and can also enhance the solubility of hydrophobic targeting molecules. This scaffold is advantageous as it can be 1. largely non-immunogenic, 2. capable of providing optimal serum persistence for different applications (tunable), 3. low in molecular mass, facilitating extravasation, tumor penetration, and renal clearance (<60 kDa), which is preferable for imaging application, 4. a platform for increasing the functional affinity of targeting molecules by using the avidity effect (2-3 targeting molecules on the same scaffold) or introducing multiple specificities, and 5. soluble in serum, rendering molecules attached to its surface also soluble.

In some embodiments, the invention provides a compound/construct comprising a) a protein scaffold, wherein the scaffold comprises Domain III, Domain IIIa, or Domain IIIb of human serum albumin or a variant thereof; b) a targeting moiety in covalent linkage to the protein scaffold; and c) a therapeutic moiety or an imaging moiety in covalent linkage to the protein scaffold. The targeting moiety is a ligand which binds a receptor of a target tissue or cell. In some embodiments, accordingly, the targeting moiety is an antibody or, more preferably, an immunologically active fragment thereof which antibody or fragment can bind a biomolecule of a target tissue or cell (e.g., a tumor specific antigen). Preferably, the antibody is an scFv diabody, a triabody, or a minibody. In some embodiments, the targeting moiety is a nucleic acid aptamer. The targeting moieties are capable of binding to a biomolecule present in a subject or on a target tissue or cell of the subject. Preferably, the biomolecule is a tumor specific antigen or other biomolecule whose presence in the targeted tissue or cell is associated with, or overexpressed, in a disease or health condition. Contemplated tumor specific antigens include, but are not limited, to CEA, CD20, HER2/neu, PSCA, PSMA, CA-125, CA-19-9, c-Met, MUC1, RCAS1, Ep-CAM, Melan-A/MART1, RHA-MM, VEGF, EGFR, integrins, and ED-B of fibronectin. Accordingly, in some embodiments, the target tissue or cell is a cancerous tissue or cell.

In some embodiments according to the invention, at least one or all of the targeting moiety, imaging moiety, or therapeutic moiety is covalently attached to the scaffold by a non-peptide linker or a non-peptide bond. In other embodiments, at least one or all of the targeting moiety, imaging moiety, or therapeutic moiety is covalently attached to the scaffold by a heterobifunctional cross linker, a homobifunctional crosslinker, a zero-length cross linker, a disulfide bond, or a physiologically cleavable cross-linker. Linkers for the targeting, imaging and therapeutic moieties are preferably from 2 to 50 atoms in length (e.g., 2 to 10, 4 to 40, 10 to 30 atoms in length). More than one targeting, imaging or therapeutic moiety may be attached to the Domain III scaffold.

In some embodiments, small peptides or other targeting moieties (aptamers, chemicals) are genetically fused or conjugated to the HSA DIII; in other embodiments, proteins (e.g., antibodies, antibody fragments, enzymes, receptor ligands, cytokines, chemokines, growth factors) are fused to the HSA DIII scaffold as the targeting moiety; or 3) nanoparticles, diamagnetic materials, Quantum dots, radionuclides, or chemical compounds may be attached to the HSA DIII scaffold as the imaging moiety.

For molecular imaging purposes, a variety of radionuclides can be attached to the protein scaffold, for detection using gamma or SPECT cameras, or PET scanners. Diamagnetic materials can be conjugated for MR imaging. For optical imaging, the HSA DIII scaffold can be fused to either a fluorescent dye, protein, or a bioluminescent enzyme (e.g., Firefly, Renilla or Gaussia luciferases).

For therapy applications, therapeutic radionucleides, cytotoxic drugs, toxins, cytokines, enzymes, or other therapeutic moieties can be linked to the targeted HSA DIII scaffold, for target specific delivery to tumors. In some embodiments, the linkage to the DIII is susceptible to cleavage under physiological conditions (e.g., enzymatic cleavage, acidic cleavage as in lysozomes).

The invention offers the advantage of providing a low or non-immunogenic human HSA Domain III proteins of lower molecular mass than HSA (e.g., 23 or 11 kDa) and which have the ability to modify or extend the serum persistence of the molecule it is attached to, to a defined degree.

In any of the above embodiments, the HSA domain III is preferably wildtype and has a mutation at H535, H510, or H464 which alters the binding of the domain to the FcRn receptor. In some embodiments, the mutation is H535A, H510A, H464A; H535A and H510A and H464A; H535A and H464A; H535A and H510A; or H510A and H464A. In some embodiments, of the above, the protein scaffold consists essentially of HSA Domain III, Domain IIIa, or Domain IIIb or polypeptides which are substantially identical to them in sequence.

In some embodiments, the therapeutic moiety of the construct is a drug. For instance, the therapeutic moiety can be a therapeutic radionucleide, a cytotoxic drug, a cytokine, a chemotherapeutic agent, a radiosensitizing agent, or an enzyme. In further embodiments, a plurality of the therapeutic moiety are covalently linked to the protein scaffold.

In other embodiments, the construct comprises the imaging agent. Suitable imaging agents include, but are not limited to, radionuclides, diamagnetic materials, paramagnetic particles, fluorophores, chromogens, quantum dots, nanoparticles, and bioluminescent enzymes. One or a plurality of imaging agents may be covalently linked to the scaffold.

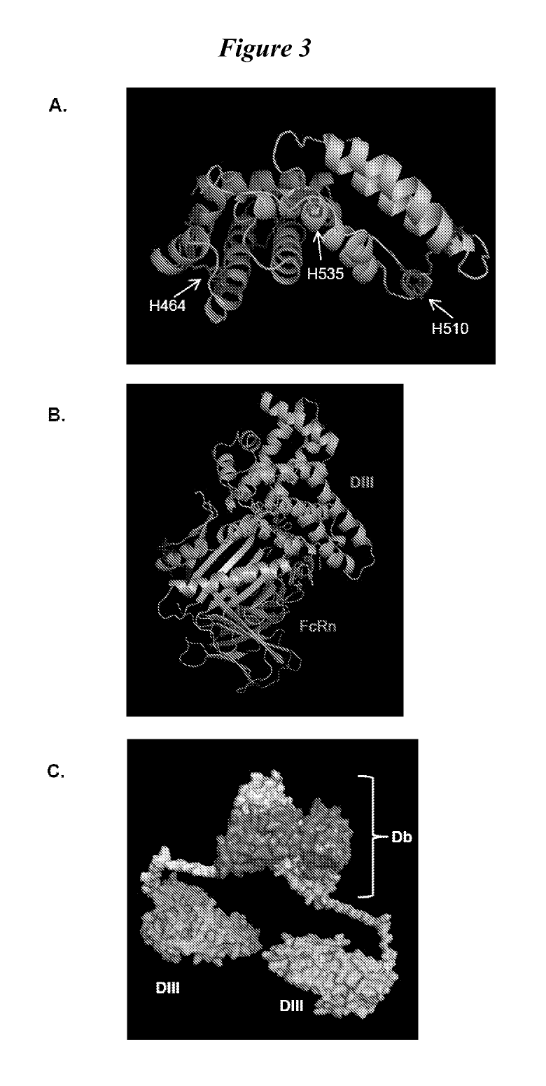

In some embodiments, the construct is mono- or multi-valent. For instance, the targeting moiety or other members of the construct (e.g., targeting moieties bound to the DIII scaffold, see FIG. 3) may themselves be mono-, di-, tri-, or multivalent with each member thereof covalently joined or linked to its HSA DIII scaffold.

In another aspect, the invention provides methods of detecting a biomolecule associated with a disease or condition in a subject by administering to a subject suspected of having, or having, the disease or condition a construct of the invention, wherein the targeting moiety of the construct binds the biomolecule and detecting the imaging agent bound to the construct. In some embodiments, the presence or absence of the disease or condition is diagnosed according to the detection. For instance, when the biomolecule is a tumor specific antigen overexpressed in cancer, the presence or absence of the cancer associated with the tumor specific antigen can be determined by administering a construct according to the invention to the subject and detecting an imaging moiety bound to the construct in the subject. The detected localization of the imaging moiety of the construct at a tumor site being indicative of the presence of the cancer. In some embodiments, the serum persistence of the construct or imaging agent is fine tuned by selecting a Domain III polypeptide which has a mutation providing an altered affinity of the Domain III (DIII) for the FcRn receptor. Radionuclides used for imaging include, but are not limited to, I-131, I-123, In-111 and Tc-99m for SPECT imaging, and F-18, I-124, Cu-64, Y-86 for PET imaging.

In still another aspect, the invention provides a method of targeted therapy of a disease or condition associated with the presence of overexpression of a biomolecule in a tissue, said method comprising administering to a subject having the disease or condition a therapeutically effective amount of the construct according to the invention wherein the targeting moiety of the construct binds the biomolecule and the therapeutic agent of the construct treats the disease or condition in the tissue or cell associated with the presence of the biomolecule. For instance, in some embodiments, the targeting moiety binds a tumor specific antigen of a cancer and the disease or condition to be treated is the cancer, and the therapeutic agent is a therapeutic radionucleide, a cytotoxic drug, a cytokine, or a chemotherapeutic agent. Therapeutic chemotherapeutic drugs that can be attached to targeted DIII include, but are not limited to: gemcitabine, doxorubicin, vincristine, topotecan, irinotecan. An example of a toxin that can be conjugated to DIII is auristatin or Pseudomonas exotoxin A. The therapeutic radionuclides include, but are not limited to, beta emitters—Y-90, Lu-177, I-131, Sm-153 and Sr-89; and alpha emitters—Ra-223, Th-227, Ac-225, At-211, Bi-212 and Bi-213. One or more therapeutic agents may be covalently attached to the DIII scaffold.

In some embodiments, therapeutic and imaging functional groups can both be attached to the same target specific DIII platform for applications such as: visualizing the targeting of the drug conjugate to the tumor/disease site, monitoring the progress of therapy by molecular imaging and determining the route of metabolic clearance.

Accordingly, the invention also provides 1) pharmaceutical or diagnostic compositions comprising the above therapeutic and imaging constructs and a physiologically acceptable excipient or carrier; 2) for the use of a therapeutic construct according to the invention, in the manufacture of a medicament for treating a disease or condition; and for the use of an imaging construct according to the invention in the manufacture of a diagnostic for detecing a disease or condition.

The invention contemplates chemically conjugating tumor targeting peptides to selected DIII platforms. Tumor bearing subjects, for instance, can be injected with ¹²⁴I (t^{1/2} 4.2 days) or ⁶⁴Cu (t_{1/2} 12.7 h) labeled proteins and their targeting of the antigen positive tumors evaluated by PET imaging. Expression of these variable region sequences on native antibody backbones, or as an scFv, triabody, diabody or minibody, labeled with radionuclide, are particularly useful in the in vivo detection of target bearing cells. Expression on such backbones or native antibody backbone can be favorable for not only targeting but also blocking the function of target biomolecules and/or killing or inhibiting the growth or proliferation of cells bearing them in vivo.

In another aspect, the invention contemplates providing a library of modified Domain III proteins having a variety of predetermined FcRn affinities for use as scaffolds in the design of targeted imaging and therapeutic constructs according to the invention. In another embodiment, the invention provides nucleic acids encoding one or more of the Domain III scaffolds and variants thereof for use according to the invention. In still further embodiments, the invention provides vectors comprising the nucleic acids operably linked to genetic regulatory factors controlling the expression of the Domain III scaffold and also provides cells containing the vectors or nucleic acids.

Three conserved histidine residues—H535, H510 and H464 in HSA DIII have been hypothesized to play a role in the HSA-FcRn binding and variants at these residues are particularly also contemplated. In order to evaluate the ability to modulate the HSA-FcRn interaction, we generated and expressed fusion proteins, consisting of the anti-CEA diabody (Db, a non-covalent dimer of two scFv; 55 kDa) and either the HSA DIII wild type (WT, non-mutated) or one of three variants, each incorporating a mutation of H535, H510 or H464 to alanine residue. Small animal PET/CT imaging of xenografted athymic mice injected with ¹²⁴I-labeled Db-DIII proteins revealed the ability of the HSA DIII to extend the serum persistence of the Db, while retaining tumor targeting. Image analysis and biodistribution studies showed that the Db-DIII WT persisted in the circulation the most with estimated mean residence time (MRT) of 56.7 h, followed by Db-DIII H535A (25 h)>H510A (20 h)>H464A (17 h) and Db (2.9 h). HSA DIII WT and variants (H535A, H510A and H464A), as well as subdomains DIIIa (amino acid residues 384 to 492; 14.2 kDa) and DIIIb (510-585; 12.2 kDa) have been generated. Their pharmacokinetic profile in blood was evaluated in vivo by injecting each ¹³¹I-labeled DIII protein intravenously in Balb/c mice. Blood was drawn from the tail at eight different time points (0-72 h) and the radioactivity was counted in a gamma well counter. The terminal serum half life (t_{1/2}β) of each protein was determined as follows: DIII WT (15.3 h), H535A (10.7 h), H464A (10.2 h), H510A (9.75 h), DIIIa (8.93 h) and DIIIb (6.87 h), compared to the entire HSA protein (17.3 h). Selected DIII proteins will be used as scaffolds for grafting or chemically conjugating tumor targeting molecules (peptides, aptamers or small chemical moieties), as well as for directly for generation of combinatorial display libraries. Target specific scaffolds with suitable pharmacokinetics for diagnostic purposes may be used in imaging applications. Alternatively, potential anti-tumor drugs could be conjugated to the targeted scaffolds with optimal characteristics for therapy and utilized in cancer treatment.

The invention also provides a docking model which indicates two more residues in DIII are important for the interaction with FcRn (i.e, glutamic acid residues E505 and E531). Accordingly, in some embodiments the invention provides variant DIII, DIIIa, or DIIIb protein scaffolds and nucleic acids, and vectors, and transduced cells comprising the nucleic acids, which have amino acid substitutions at position E505 and/or E531 and are otherwise substantially identical or identical to the Domain III, Ma, or Mb sequence of HSA. In some embodiments, either or both these residues are substituted with aspartic acid, in other embodiments, either or both of these amino acids are substituted with an uncharged amino acid, and in still further embodiments, either or both of E505 and E531 are substituted by alanine or glycine. In yet other embodiments, the substitution is E505D, A, G, I, V, or L or E531D A, G, I, V, or L substitution which perturbs DIII binding to FcRn and thus modulates the circulation half life of the target specific DIII imaging or therapeutic agent.

Targeting moieties may be any molecule capable of binding to a target biomolecule. In some embodiments, the target molecule is a tumor specific antigen present on the external surface of a cell. A targeting moiety can be an antibody, or more preferably, a fragment of an antibody which has affinity for the molecule recognized by the antibody. In preferred embodiments, the antibody is an scFv, a diabody, a minibody, or a triabody. In some embodiments, the targeting moiety is an nucleic acid aptamer or a small peptide (e.g., 5 to 30 amino acids, 2 to 20 amino acids in length) which is capable of binding the biomolecule. Preferably, the targeting moiety has a high affinity for the biomolecule and has a K_{d} of less than 100 nM, 30 nM, 10 nM, or 1 nM. In addition, use of multiple targeting moieties (2, 3, 4 or more), of these or lower affinities for a target biomolecule, per scaffold can enhance binding to a target cell via an avidity effect.

The term “imaging agent or moiety” is used herein to refer to agents or moieties that are capable of providing a detectable signal, either directly or through interaction with additional members of a signal producing system. Preferably, the signal is capable of being detected externally when generated by a construct within the body of a subject.

A “therapeutic moiety” refers to an agent which is useful in treating a disease or condition or having some other intended benefit to the subject, targeted tissue and/or cell. A therapeutic moiety can be a therapeutic drug, hormone, cytokine, interferon, antibody or antibody fragment, nucleic acid aptamer, enzyme, polypeptide, toxin, cytotoxin, or chemotherapeutic agent. A therapeutic moiety can be a radiation sensitizer.

The linkers used to join the targeting moiety, imaging moiety, or therapeutic moiety to the scaffold may comprise a covalent bond or a chain of atoms from 1 to 100 atoms in length or longer. Linkers may comprise carbon, nitrogen, sulfur, or oxygen atoms in the chain. Carbon chains are specifically contemplated (e.g., from about 5 to about 50 carbons). A linker may comprise nucleic acids or amino acids. Examples of carbon chains as linkers include, but are not limited to, an alkyl, alkene, or aldehyde. The carbon chain may be one or more of substituted, un-substituted, unbranched, or branched. A linker may comprise a length of from about 5 to about 50 nanometers, 3 to 30 nm, and more preferably, from about 5 to about 10 nm. Examples of linkers may include, but are not limited to, carbon chains having a length of from about 10 carbons to about 20 carbons. Polyalkylene glycol (e.g., PEG) linkers are also contemplated. Linkers can include a non-peptide bond. Linkers include, but are not limited to, heterobifunctional cross linker, a homobifunctional crosslinker, a zero-length cross linker, a disulfide bond, or a physiologically cleavable cross-linker. Linkers for the targeting, imaging and therapeutic moieties are preferably from 2 to 80 atoms in length (e.g., 2 to 10, 4 to 40, 10 to 30, 2 to 50 atoms in length). Fusion proteins of the domain III and at least one of the targeting agent, imaging agent, or therapeutic agent are also contemplated when the fused agent is a polypeptide. It is also contemplated that the targeting, imaging and therapeutic agents may each not be joined to the scaffold as a fusion protein or are not joined to the scaffold by another amino acid or by a peptide bond.

Imaging agents and therapeutic moieties may be conjugated directly to the DIII protein scaffold using conventional methods that are well known in the art. Radioactive and non-radioactive labels are commonly employed (For a review of enzymatic, photochemical, and chemical methods for labeling nucleic acids and proteins see, Bioconjugate Techniques, 2nd Edition By Greg T. Hermanson, Published by Academic Press, Inc., 2008, 1202 pages.)

Aptamers are oligonucleic acid molecules that bind to a specific target molecule. Aptamers are usually created by selection operating upon large random sequence pools. By methods well known in the art, nucleic acid aptamers can be obtained by repeated rounds of in vitro selection or equivalently, SELEX (systematic evolution of ligands by exponential enrichment) to bind to various molecular targets such as small molecules, proteins, nucleic acids, and even cells, and tissues. As well known in the art, nucleic acid aptamers can be generated by in vitro screening of complex nucleic-acid based combinatorial shape libraries (e.g., >10¹⁴ shapes per library) employing a process termed SELEX (see, U.S. patent publication no. 20090004667 which is incorporated herein by reference). SELEX is an iterative process in which a library of randomized pool of RNA sequences is incubated with a selected protein target. Interacting RNA is then partitioned from non-binding RNA and subsequently amplified through reverse transcription followed by amplification via polymerase chain reaction (RT/PCR). A DNA template can be used to create an enriched RNA pool through in vitro transcription with a mutant T7 RNA polymerase that allows for the incorporation of 2′ fluoro-modified pyrimidines. These modifications render the RNA more nuclease resistant. The steps leading to the creation of the enriched RNA pool are referred to as a “selection round”. The selection rounds against a protein target are typically continued until a plateau in binding affinity progression had been reached. Individual clones may then be isolated from the pool and sequenced. Aptamers can provide molecular recognition properties rivaling or exceeding that of antibodies. In addition to their specific recognition, aptamers offer advantages over antibodies. They can be engineered completely in a test tube and are readily manufactured by chemical synthesis. Aptamers also possess desirable storage properties and solubility properties and elicit comparatively little or no immunogenicity in therapeutic applications. An aptamer for use according to the invention can be a nucleic acid which binds with high affinity (e.g., having a K_{d} less than 100 nM, 10 nM, or 1 nM) to CEA, CD20, HER2/neu, PSCA, PSMA, CA-125, CA-19-9, c-Met, MUC1, RCAS1, Ep-CAM, Melan-A/MART1, RHA-MM, VEGF, EGFR, integrins, and ED-B of fibronectin. Aptamers are preferably from 10 to 30, 10 to 20, or 15 to 25, nucleic acids in length.

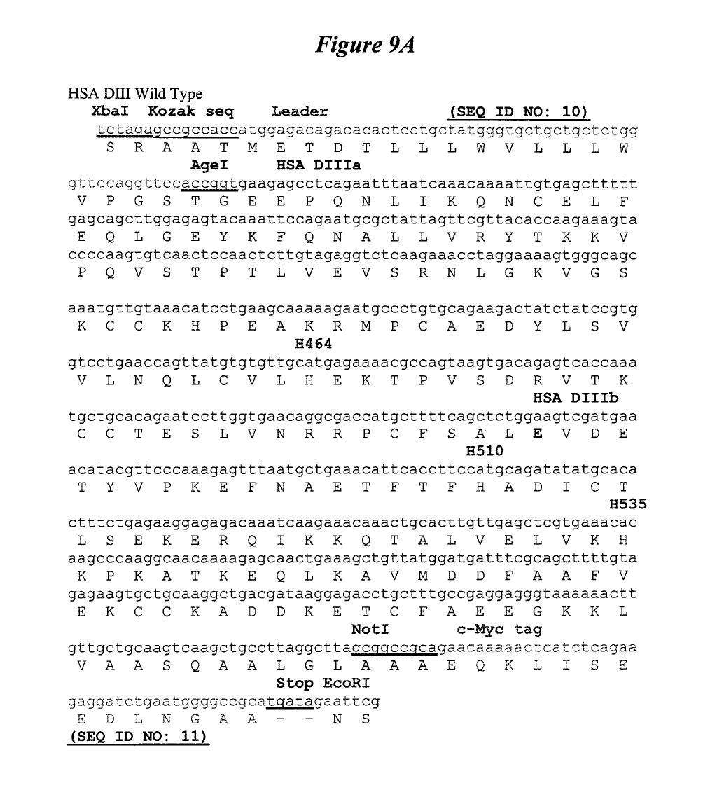

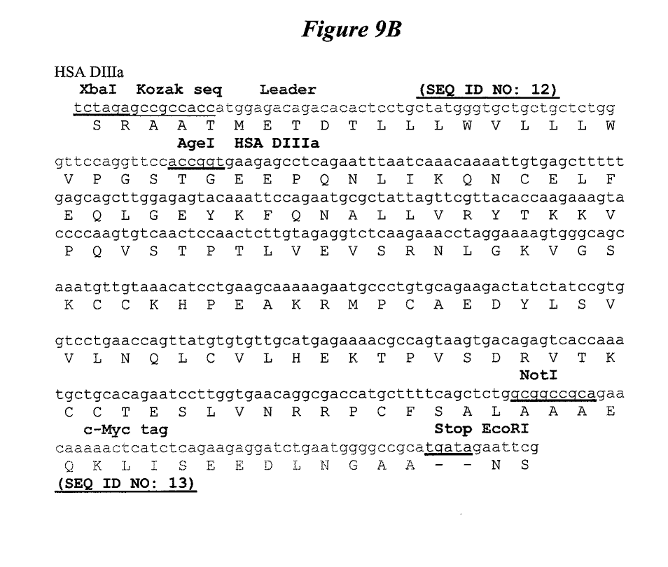

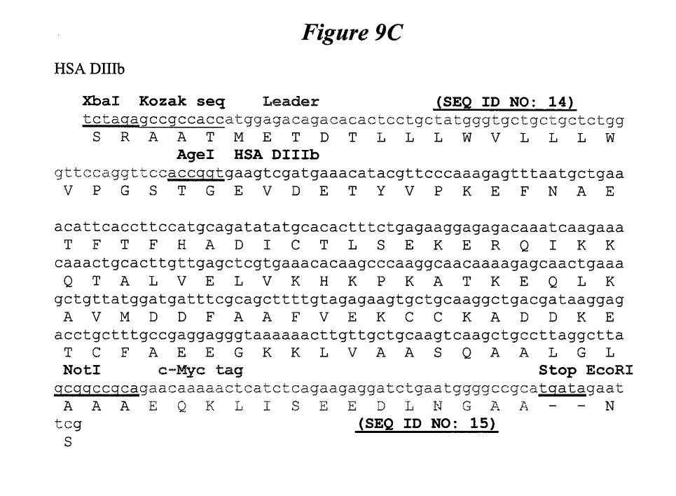

The amino acid sequence of Domain III according to the invention is that of a HSA Domain III, IIIa, or IIIb (see, FIGS. 9a, b, c, respectively) or a sequence which is substantially identical thereto. Domain III 1) comprises, consists of, or consists essentially of an amino acid sequence that has greater than about 90%, preferably 91%, 92%, 93%, 94%, 95%, 96%, 97%, 98% 99% or 100% amino acid sequence identity, preferably over the full sequence or over a region of at least about 15, 20, 25, 50, 75, 100, 125, 150 or more amino acids, to a polypeptide of FIGS. 9a, 9b, or 9c and can bind the FcRn (Brambell) receptor. Domain III (amino acids residues 384 to 585) has two subdomains—DIIIa (amino acid residues 384 to 492) and DIIIb (amino acid residues 510-585). (see, Sugio et al, Protein Engineering, Vol. 12, No. 6, 439-446, June 1999) which is incorporated herein by reference with regard to HSA sequence and structure). In some embodiments, the Domain III of the claims is a polypeptide comprising, consisting of, or consisting essentially of Domain III, Domain IIIa or Domain IIIb of HSA and their H535, H510, or H464 variants disclosed herein.

A Domain III, Domain IIIa, or Domain IIIb according to the invention may be a conservatively modified variant of a polypeptide of FIG. 9a, b, or c, respectively. In preferred embodiments, the variant has an altered affinity for the FcRn (Brambell) receptor which fine tunes its serum persistence. In some embodiments, one or more of the histidine residues at position H535, H510, H464 of these domains is deleted or replaced by another basic or non-basic amino acid. In some embodiments, the Domain III sequence has a substitution, or only a substitution, which is one or more of H535A or G, H510A or G, H464A or G. In some further embodiments, the substitution is one, two, or three of H535A, H510A, H464A. In other embodiments, the substitution is any one or more of H535V, I, or L; H510 V, L, or I; or H464 V, L, or I. In further embodiments, other conservative substitutions (1, 2, 3, 4, or more) are made at other positions of the domain III, IIIa, or Mb scaffold. A HSA sequence is also set forth in GenBank: AAA98797.1.

The terms “polypeptide,” “peptide” and “protein” are used interchangeably herein to refer to a polymer of amino acid residues. Methods for obtaining (e.g., producing, isolating, purifying, synthesizing, and recombinantly manufacturing) polypeptides are well known to one of ordinary skill in the art.

The term “amino acid” refers to naturally occurring and synthetic amino acids, as well as amino acid analogs and amino acid mimetics that function in a manner similar to the naturally occurring amino acids. Preferred amino acids are the naturally occurring amino acids as found in humans. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Diabodies, first described by Hollinger et al., PNAS (USA) 90(14): 6444-6448 (1993), may be constructed using heavy and light chains disclosed herein, as well as by using individual CDR regions disclosed herein. Typically, diabody fragments comprise a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{H} and V_{I}, domains of another fragment, thereby forming two antigen-binding sites. Triabodies can be similarly constructed with three antigen-binding sites. An Fv fragment contains a complete antigen-binding site which includes a V_{L} domain and a V_{H} domain held together by non-covalent interactions. Fv fragments embraced by the present invention also include constructs in which the V_{H} and V_{L} domains are crosslinked through glutaraldehyde, intermolecular disulfides, or other linkers. The variable domains of the heavy and light chains can be fused together to form a single chain variable fragment (scFv), which retains the original specificity of the parent immunoglobulin. Single chain Fv (scFv) dimers, first described by Gruber et al., J. Immunol. 152(12):5368-74 (1994), may be constructed using heavy and light chains disclosed herein, as well as by using individual CDR regions disclosed herein. Many techniques known in the art can be used to prepare the specific binding constructs of the present invention (see, U.S. Patent Application Publication No. 20070196274 and U.S. Patent Application Publication No. 20050163782, which are each herein incorporated by reference in their entireties for all purposes, particularly with respect to minibody and diabody design).

Bispecific antibodies can be generated by chemical cross-linking or by the hybrid hybridoma technology. Alternatively, bispecific antibody molecules can be produced by recombinant techniques (see: bispecific antibodies). Dimersation can be promoted by reducing the length of the linker joining the VH and the VL domain from about 15 amino acids, routinely used to produce scFv fragments, to about 5 amino acids. These linkers favor intrachain assembly of the VH and VL domains. A suitable short linker is SGGGS (SEQ ID NO: 1) but other linkers can be used. Thus, two fragments assemble into a dimeric molecule. Further reduction of the linker length to 0-2 amino acids can generate trimeric (triabodies) or tetrameric (tetrabodies) molecules.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., in Monoclonal Antibodies and Cancer Therapy, Alan R. Liss, Inc., pp. 77-96 (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher et al., J. Biol. Chem. 273(52):35095-35101 (1998).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ, is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

A “label” or a “detectable moiety” or “imaging agent or moeity” is a compound detectable by spectroscopic, photochemical, biochemical, immunochemical, chemical, radiologic, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide. Preferred imaging agents or moieties are magnetic, fluorescent, or radioactive. Methods of detecting the signal generated by the labels in vitro and in vivo are well known in the art.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

### Compositions.

When used for pharmaceutical purposes with regard to the invention, the constructs according to the invention are typically formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al., Biochemistry 5:467 (1966). The compositions can additionally include a stabilizer, enhancer, or other pharmaceutically acceptable carriers or vehicles. A pharmaceutically acceptable carrier can contain a physiologically acceptable compound that acts, for example, to stabilize the nucleic acids or polypeptides of the invention and any associated vector. A physiologically acceptable compound can include, for example, carbohydrates, such as glucose, sucrose or dextrans; antioxidants, such as ascorbic acid or glutathione; chelating agents; low molecular weight proteins or other stabilizers or excipients. Other physiologically acceptable compounds include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, Mack Publishing Company, Philadelphia, Pa., 17th ed. (1985).

The pharmaceutical compositions according to the invention comprise a therapeutically effective amount of a construct according to the invention according to the invention and a pharmaceutically acceptable carrier. By “therapeutically effective dose or amount” herein is meant a dose that produces effects for which it is administered (e.g., treatment or prevention of a retinal detachment). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). The construct, if a salt, is formulated as a “pharmaceutically acceptable salt.”

A “pharmaceutically acceptable salt” or to include salts of the active compounds which are prepared with relatively nontoxic acids or bases, according to the route of administration. When inhibitors of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Aside from biopolymers such as nucleic acids and polypeptides, certain compounds of the present invention possess asymmetric carbon atoms (optical centers) or double bonds; the racemates, diastereomers, geometric isomers and individual isomers are all intended to be encompassed within the scope of the present invention. In preferred embodiments, wherein the compound comprises amino acids or nucleic acids, the amino acids and nucleic acids are each the predominant naturally occurring biological enantiomer.

The compositions for administration will commonly comprise an agent as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Suitable formulations for use in the present invention are found in Remington: The Science and Practice of Pharmacy, 20th Edition, Gennaro, Editor (2003) which is incorporated herein by reference. Moreover, for a brief review of methods for drug delivery, see, Langer, Science 249:1527-1533 (1990), which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

For injection, the compounds of the present invention can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the inhibitors for use according to the invention can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

The pharmaceutical compositions can be administered in a variety of dosage forms and amounts depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

Pharmaceutical formulations, particularly, of the constructs according to the present invention can be prepared by mixing the construct having the desired degree of purity with optional pharmaceutically acceptable carriers, excipients or stabilizers. Such formulations can be lyophilized formulations or aqueous solutions. Acceptable carriers, excipients, or stabilizers are nontoxic to recipients at the dosages and concentrations used. Acceptable carriers, excipients or stabilizers can be acetate, phosphate, citrate, and other organic acids; antioxidants (e.g., ascorbic acid) preservatives low molecular weight polypeptides; proteins, such as serum albumin or gelatin, or hydrophilic polymers such as polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g. Zn-protein complexes); and/or non-ionic surfactants. In some embodiments, the construct can be formulated at a concentration of between 0.5-200 mg/ml, or between 10-50 mg/ml.

The compositions containing the constructs the invention can be administered for diagnostic, therapeutic or prophylactic treatments. In therapeutic applications, compositions are administered to a patient in a “therapeutically effective dose.” Single or multiple administrations of the compositions may be administered depending on the dosage and frequency as required and tolerated by the patient. A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

As used herein, the term “carrier” refers to a typically inert substance used as a diluent or vehicle for an active agent to be applied to a biological system in vivo or in vitro. (e.g., drug such as a therapeutic agent). The term also encompasses a typically inert substance that imparts cohesive qualities to the composition.

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically or physiologically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate.

### Methods of Treatment

The terms “treating” or “treatment” includes:

(1) preventing the disease, i.e., causing the clinical symptoms of the disease not to develop in a mammal that may be exposed to the organism but does not yet experience or display symptoms of the disease,

(2) inhibiting the disease, i.e., arresting or reducing the development of the disease or its clinical symptoms. This includes reducing the extent of the detachment observed or the numbers of subjects or risk of a subject having a detachment.

(3) relieving the disease, i.e., causing regression of the disease or its clinical symptoms.

The constructs for used according to the invention may be administered by any route of administration (e.g., intravenous, topical, intraperitoneal, parenteral, oral, intravaginal, rectal, ocular, intravitreal and intraocular). They may be administered as a bolus or by continuous infusion over a period of time, by intramuscular, intraperitoneal, subcutaneous, oral, topical, or inhalation routes. Intravenous or subcutaneous administration of the antibody is preferred. The administration may be local or systemic. They may be administered to a subject who has been diagnosed with the subject disease, a history of the disease, or is at risk of the disease.

### EXAMPLES

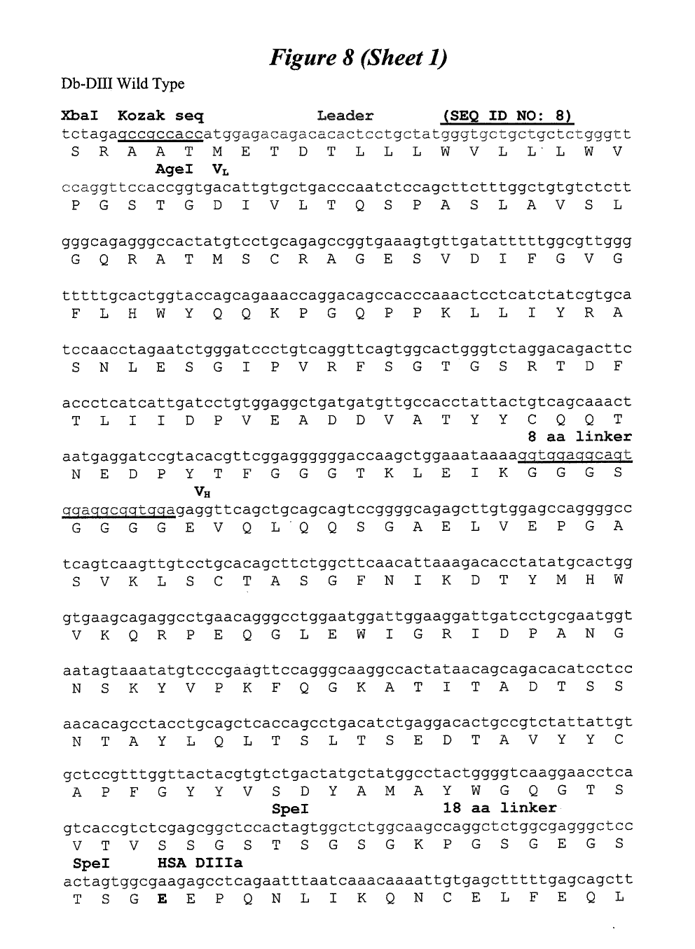

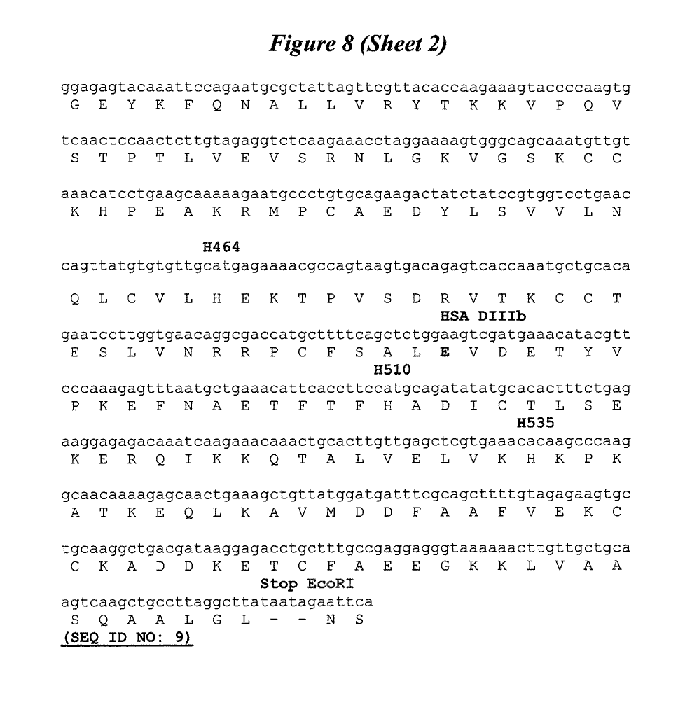

The following examples are offered to illustrate, but not to limit the claimed invention. While the invention is exemplified with a fusion protein of CEA and a Domain III scaffold (see, FIG. 8), other methods of conjugating the scaffold to the targeting agent and or imaging and therapeutic agents are contemplated.

### Example 1

We tested fusion proteins consisting of a well studied antibody fragment targeting carcinoembryonic antigen (CEA) and either the HSA DIII wild type (WT, non-mutated) or one of three HSA DIII variants, each incorporating a mutation of H535, H510 or H464 to alanine residue. Xenografted athymic nude mice were injected with 1241-labeled Db-DIII or Db proteins, and serial small animal PET/CT imaging studies were performed to evaluate the ability of the HSA DIII to modulate the serum persistence of the Db in vivo. In addition, we were able to draw conclusions about the relative importance of the H535, H510 and H464 residues for FcRn binding and circulation persistence of albumin.

### Materials and Methods

### Generation of Db-DIII Constructs

HSA DIII genes were amplified by polymerase chain reaction (PCR) using commercial HSA cDNA (OriGene Technologies, Rockville, Md.) as a template and primers introducing 5′ SpeI and 3′ EcoRI restriction sites. The primer sequences were as follows:

Forward: SpeI-DIII:
(SEQ ID NO: 2)
5′-CCACTAGTGGCGAAGAGCCTCAGAATTTAATC-3′
Reverse: DIII-EcoRI:
(SEQ ID NO: 3)
5′-GAGAATTCTATTATAAGCCTAAGGCAGCTTGAC-3′

Mutations of histidine residues H535, H510 or H464 to alanine in the DIII, were introduced by site directed mutagenesis, using a Quick-Change mutagenesis kit (Stratagene, La Jolla, Calif.) with the appropriate mutagenesis primers (only forward primers are listed): H464A (exchanging histidine residue in position 464 with an alanine residue)

(SEQ ID NO: 4)
5′-CTGAACCAGTTATGTGTGTTGGCTGAGAAAACGCCAGTAAGTGAC-
3′
H510A
(SEQ ID NO: 5)
5′-GTTTAATGCTGAAACATTCACCTTCGCTGCAGATATATGCACAC-3′
H535A
(SEQ ID NO: 6)
5′-CTGCACTTGTTGAGCTCGTGAAAGCCAAGCCCAAGGCAAC-3′

The complete DIII (WT, H535A, H510A and H464A) genes were cloned in pCR2.1-Topo vector (Invitrogen, Carlsbad, Calif.) and then transferred into the pUC18 vector (New England Biolabs, Beverly, Mass.), already containing the anti-CEA Db (Wu et al., 1999). The entire Db-DIII genes were excised from the pUC18 vector and ligated into the pEE12 mammalian expression vector (Bebbington et al., 1992), using XbaI and EcoRI sites.

### Expression, Selection and Purification

NS0 murine myeloma cells (Sigma-Aldrich, St. Louis, Mo.) were maintained in non-selective glutamine-free Dulbecco's modified Eagle's Medium (DME/High Modified; SAFC Biosciences, Lenexa, Kans.), supplemented with 5% heat inactivated, dialyzed fetal bovine serum (FBS; Omega Scientific Inc., Tarzana, Calif.), 1% v/v of 200 mM L-glutamine (Mediatech, Inc., Manassas, Va.) and 1% v/v of Penicillin-Streptomycin (10,000 IU/ml penicillin, 10,000 μg/ml streptomycin; Mediatech Inc.). 1×10⁷NS0 cells in log growth phase were transfected by electroporation with 10 μg of pEE12-Db-DIII DNA, linearized by digestion with SalI (New England Biolabs, Ipswich, Mass.), as previously described (Kenanova et al., 2005).

Db-DIII production was assayed by ELISA and confirmed by Western blot. For ELISA, Protein A (Thermo Fisher Scientific, Rockford, Ill.) was used to capture the Db-DIII proteins. Alkaline phosphatase (AP)-conjugated anti-mouse Fab-specific antibody (Sigma-Aldrich) served for detection in both ELISA and Western blot. Transfected NS0 cells were maintained in selective glutamine-free DME/High Modified medium (SAFC Biosciences), supplemented with 5% heat inactivated, dialyzed FBS (Omega Scientific Inc.), 2% v/v of 50×GS supplement (SAFC Biosciences) and 1% v/v Penicillin-Streptomycin (Mediatech Inc.). Selected clones, expressing high amounts of Db-DIII proteins, were gradually expanded into triple flasks (Nunclon, Rochester, N.Y.), containing 300 ml selective media, supplemented with 2% heat inactivated, dialyzed FBS (Omega Scientific Inc.) and 1% v/v Penicillin-Streptomycin (Mediatech Inc.).

When cultures reached terminal state (˜3 weeks), harvested supernatants were centrifuged, filter sterilized and concentrated, using a Lab Scale tangential flow filtration (TFF) system (Millipore, Billerica, Mass.) with a 30,000 Da molecular weight cut-off (mwco) filter. Db-DIII proteins were purified on a Protein A column (Thermo Fisher Scientific, Inc.), using an AKTA Purifier (GE Healthcare, Piscataway, N.J.). The bound protein was eluted at 15% of 0.2 M Citrate buffer (pH 2.1) in 1×PBS and pH was immediately neutralized by adding 80% v/v of 1 M Tris base (pH 8.2) directly to the eluted proteins. Fractions containing pure Db-DIII protein were pooled, dialyzed against 1×PBS, and concentrated by Vivaspin 20 (mwco: 30,000; Sartorius Stedim Biotech Gmbh, Goettingen, Germany). The final concentration of purified Db-DIII proteins was determined by A₂₈₀, using an extinction coefficient c=1.5.

### Characterization of Db-DIII Proteins

Purified Db-DIII proteins were analyzed by sodium dodecyl sulfate polyacrylamide gel electrophoresis (SDS-PAGE) under non-reducing (NR) and reducing (R) conditions, Western blot, mass spectrometry and size exclusion chromatography. To reduce the protein, 1M dithiothreitol (DTT) was added to a final concentration of 0.2 M. For the SDS-PAGE, 4-20% gradient Tris-HCl ready gels (Bio-Rad Laboratories, Hercules, Calif.) were run and developed in Instant Blue Coomassie-based solution (Expedion Protein Solutions, Cambridge, UK). Detection of the Db-DIII proteins in Western blots was accomplished with AP-conjugated goat anti-mouse Fab-specific mAb (Sigma-Aldrich) using nitro blue tetrazolium (NBT) and 5-bromo-4-chloro-3-indolyl-phosphate (BCIP) (Promega, Madison, Wis.) AP substrates, or horse radish peroxidase (HRP)-conjugated Protein L (Sigma-Aldrich) developed with the 4-chloro-1-naphthol/3,3′-diaminobenzidine (CN/DAB) substrate kit (Thermo Scientific, Rockfort, Ill.).

Mass spectrometry using an LTQ-FT Ultra Linear Ion Trap Fourier Transform Ion Cyclotron Resonance (FT-ICR) mass spectrometer (Thermo Fisher) was performed to confirm the identity of the purified proteins. Briefly, Db-DIII proteins were isolated following an in-gel trypsin digestion procedure. Nano-liquid chromatography with tandem mass spectrometry (nLC-MSMS) and collisionally activated dissociation (CAD) fragmentation was performed on an LTQ-FT (Thermo Fisher) integrated with an Eksigent nano-LC. Spectra were searched against the most up-to-date International Protein Index database (Version 3.54 with 39,925 entries) using the Mascot (Matrix Science, UK) and Sequest (Thermo Fisher) programs. The results were filtered with a strict score filtering criterion and a 10 ppm mass resolution filter. Identified peptides were also matched to the Db-DIII sequence.

Determination of Db-DIII protein purity after purification, Db-DIII protein conformation under native, non-denaturing conditions (1×PBS, pH 7.4), and estimation of molecular size was accomplished through size exclusion chromatography using a Superdex 200 HR 10/30 column (GE Healthcare).

Computer models of DIII and Db-DIII molecules were generated using the PyMOL software (DeLano Scientific). Additionally, modeling of protein docking between HSA and DIII was accomplished using the ZDOCK-FFT algorithm (Chen et al., 2003), available on a public server (http://zlab.bu.edu/˜rong/dock).

### Radioiodination of Db-DIII Fusion Proteins, Xenograft Imaging and Biodistribution

Purified Db-DIII WT, H535A, H510A and H464A were radioiodinated with the positron emitter ¹²⁴I (sodium iodide in 0.02 M NaOH; IBA Molecular, Sterling, Va.) using the Iodogen method as previously described (Olafsen et al., 2006). Labeling reactions (0.114-0.130 ml) contained 0.1 mg purified protein and 12.9-18.0 MBq Na¹²⁴I. Labeling efficiency was measured by instant thin layer chromatography (ITLC) using the monoclonal antibody ITLC strips kit (Biodex Medical Systems, Shirly, N.Y.), as previously described (Olafsen et al., 2006).

For in vivo studies, 7 to 8 week old athymic nude mice (Charles River Laboratories, Wilmington, Mass.) were injected subcutaneously in the left shoulder region with 1−5×10⁶ CEA-positive LS174T human colon carcinoma cells (American Type Culture Collection, Manassas, Va.) and in the right shoulder area with approximately the same number of CEA-negative C6 rat glioma cells (ATCC). Tumor masses were allowed to develop for an average of 10 days and reached a maximum of 200 mg weight. Four tumor bearing mice per construct were injected in the tail vein with 3.9-5.4 MBq ¹²⁴I-labeled Db-DIII or Db in saline/1% HSA.

At five different time points (4 h, 20 h, 28 h, 44 h and 51 h), the injected mice were anesthetized using 2% isoflurane, placed on the bed, and imaged for 10 min. A 10 min CT scan was completed following the final PET scan at 51 h. All imaging experiments utilized the Focus 220 small animal PET (Siemens Preclinical Solutions, Knoxyille, Tenn.) and the small animal CAT II (Concorde Microsystems, Knoxyille, Tenn.) scanners. Following the last scan (51 h), mice were euthanized. Blood, tumors (LS174T and C6), liver, spleen, kidneys, lungs and carcass were collected, weighed, and counted in a Wallac WIZARD Automatic Gamma Counter (PerkinElmer Life and Analytical Sciences Inc., Wellesley, Mass.). After decay correction, the percent injected dose per gram (% ID/g) for each tissue/organ was calculated, incorporating a correction for the labeling efficiency of each protein and a standard error (SE).

### Image Analysis and Statistics

All images were reconstructed using a filtered back projection (FBP) algorithm (Defrise et al., 1997) and displayed by the AMIDE software (Loening and Gambhir, 2003). The same color threshold was applied to all images. Regions of interest (ROI; ellipsoid, 0.4 mm depth, n=4) were drawn in the area of the CEA-positive tumor and in a low-activity, soft tissue region of the lower body (muscle). Tumor-to-soft tissue (T:ST) ratios were determined for individual mice and averaged for each time point and construct. ROIs (n=4) were drawn over the heart on each image and % ID/g of blood was calculated by the AMIDE software after entering the injected dose in MBq and cylinder factor in MBq/cc/image units as input functions. The ADAPTII software package was used to calculate the mean residence time (MRT) of each protein from its blood activity curve (D′ argenio and Schumitzky, 1979). SE was calculated for all ratios and % ID/g values, and expressed graphically (error bars). All T:ST ROI ratios and blood activity curves, respectively, were compared for significant difference using an unpaired Student t test. A 2-tailed P value of less than or equal to 0.05 was considered statistically significant.

### Results

### Production and Biochemical Characterization of Db-DIII Proteins

a. Generation, Expression and Purification

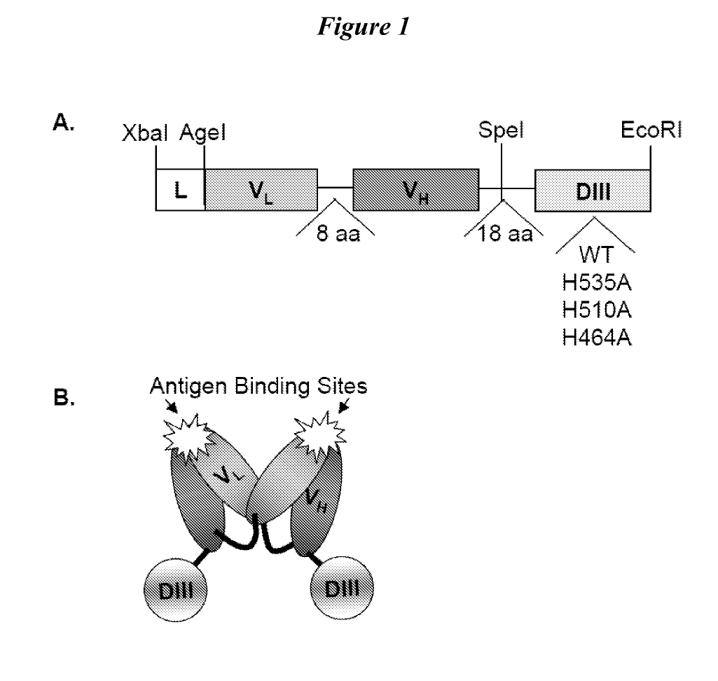

The Db-DIII construct is approximately 1.4 kilobase pairs long, flanked by XbaI and EcoRI restriction sites (FIG. 1A). The engineered Db-DIII molecules were expressed at 10-16 μg/ml in terminal cultures of transfected NS0 cells, as determined by ELISA. Although Protein L was capable of binding the Db-DIII proteins, capture by Protein A was more efficient. Therefore, Protein A affinity chromatography was selected for purification. Because the Db is a non-covalent dimer of two scFv molecules, each Db molecule has two DIII proteins attached to its C-termini, resulting in a fusion protein of approximately 101 kDa calculated molecular mass (FIG. 1B).

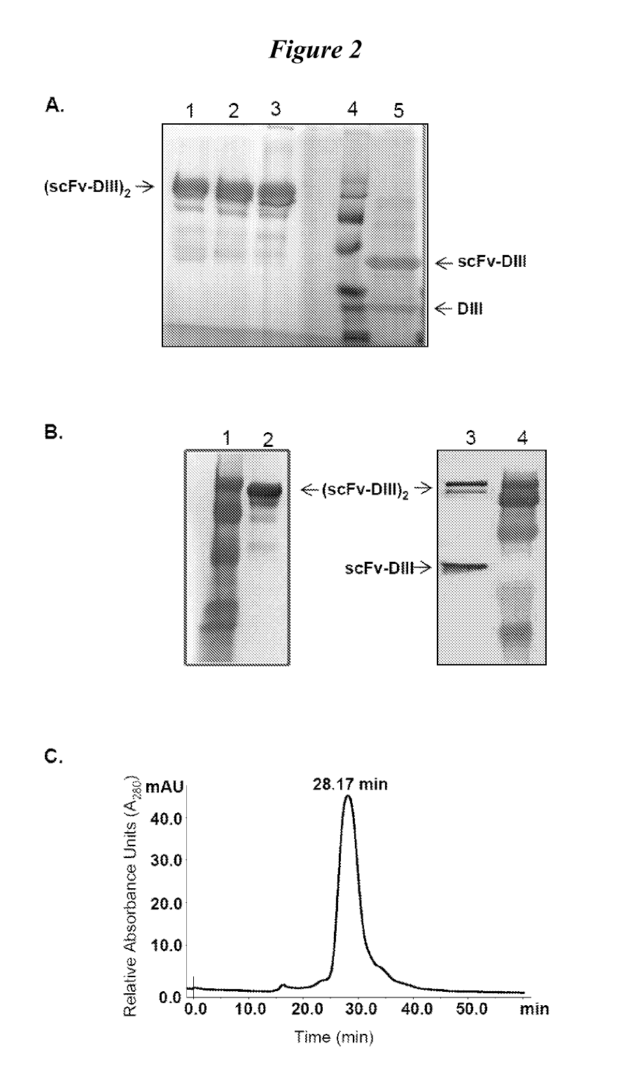

b. SDS-PAGE and Western Blot Purified Db-DIII WT and variants were analyzed by SDS-PAGE under NR and R conditions (FIG. 2A). Db-DIII proteins produced a major band corresponding to their predicted molecular mass of approximately 101 kDa under NR conditions (FIG. 2A, lanes 1, 2 and 3). Two weaker bands of lower molecular mass were also noted both on the SDS-PAGE Coomassie stained gel (FIG. 2A, lanes 1, 2 and 3) and the Western blot, probed with an anti-mouse Fab specific antibody (FIG. 2B, lane 2). When reduced, the major band [(scFv-DIII)₂; 101 kDa] splits down to two bands corresponding to a scFv-DIII fragment (˜48 kDa) and a DIII molecule (˜23 kDa) (FIG. 2A, lane 5). An attempt to detect the DIII portion of the fusion protein with a polyclonal anti-HSA antibody was not successful, therefore HRP-conjugated Protein L, binding to the Db component of Db-DIII protein, was used instead in the Western blot (FIG. 2B, lane 3). Only the upper [(scFv-DIII)₂; 101 kDa] and the middle (scFv-DIII; 48 kDa) bands were detected. Therefore, the lower band of about 23 kDa on the reduced protein SDS-PAGE gel (FIG. 2A, lane 5) should represent the DIII domain alone. In order to confirm that the purified protein was indeed Db-DIII, mass spectrometry was employed. Several peptides matching Db or DIII amino acid sequences were determined, confirming the identity of the protein (data not shown).
c. Size Exclusion Chromatography

Size exclusion chromatography showed that Db-DIII WT (101 kDa) was eluted as a single peak with elution time of 28.17 min (FIG. 2C). Under the same conditions, the Db-DIII H535A, H510A and H464A proteins were characterized by an average elution time of 28.2 min, and no aggregation or multimerization was detected. Integration of the size exclusion chromatography peaks revealed about 98% protein purity after a single step of Protein A affinity column purification.

d. Computer Modeling of HSA DIII, DIII-FcRn Interaction and Db-DIII

A structural model of HSA DIII was generated based on the crystal structure of HSA (Sugio et al., 1999) (FIG. 3A). DIII is comprised of ten α-helices (six in DIIIa and four in DIIIb) connected to each other by loops. Residues H464 (in DIIIa), H535 and H510A (both in DIIIb) are depicted. A docking model of DIII and FcRn was also generated (FIG. 3B), using the crystal structures of HSA and FcRn (Martin et al., 2001). The ZDOCK algorithm was biased towards interactions that included the FcRn residues H161 and H166 (Andersen et al., 2006), and the HSA DIII H535, H510 and H464. It produced eleven candidate structures. These structures were sorted and analyzed using PyMOL for potential strong pH dependent binding. The overall impression from the analysis was that the conserved aromatic residues surrounding FcRn residues H166 and H161 are likely to make contact with two of the DIII H510 and H535 residues, as they did in a majority of the predicted structures. FcRn H166 and H161 seemed likely to interact with glutamic acid residues on DIII that would increase in affinity when the histidines were protonated in low pH environments. In addition, FcRn residues D102 and N101 interacted in many of the proposed structures and are likely to play a role. The tenth resultant structure provided by ZDOCK was deemed the most likely to exhibit strong pH dependent binding. This structure contained potential interactions between DIII H535 and FcRn F157; DIII H510 and FcRn W51 and Y60; DIII H464 and either FcRn D101 and N₁₀₂ or K123; FcRn H166 and DIII E505; and FcRn H161 and DIII E531. Finally, a model of the Db-DIII molecule was created using the crystal structure of the T84.66 diabody (Carmichael et al., 2003) (FIG. 3C). Two DIII molecules are attached to each dimeric diabody through 18 amino acid linkers, which should produce a relatively flexible molecular structure.

### Radiolabeling and Murine Xenograft Imaging Studies

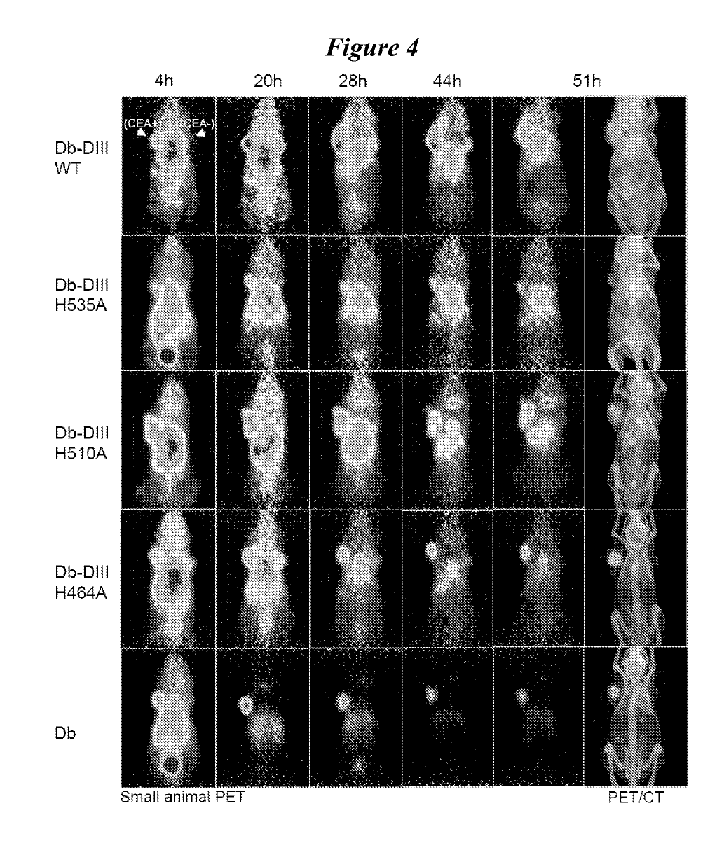

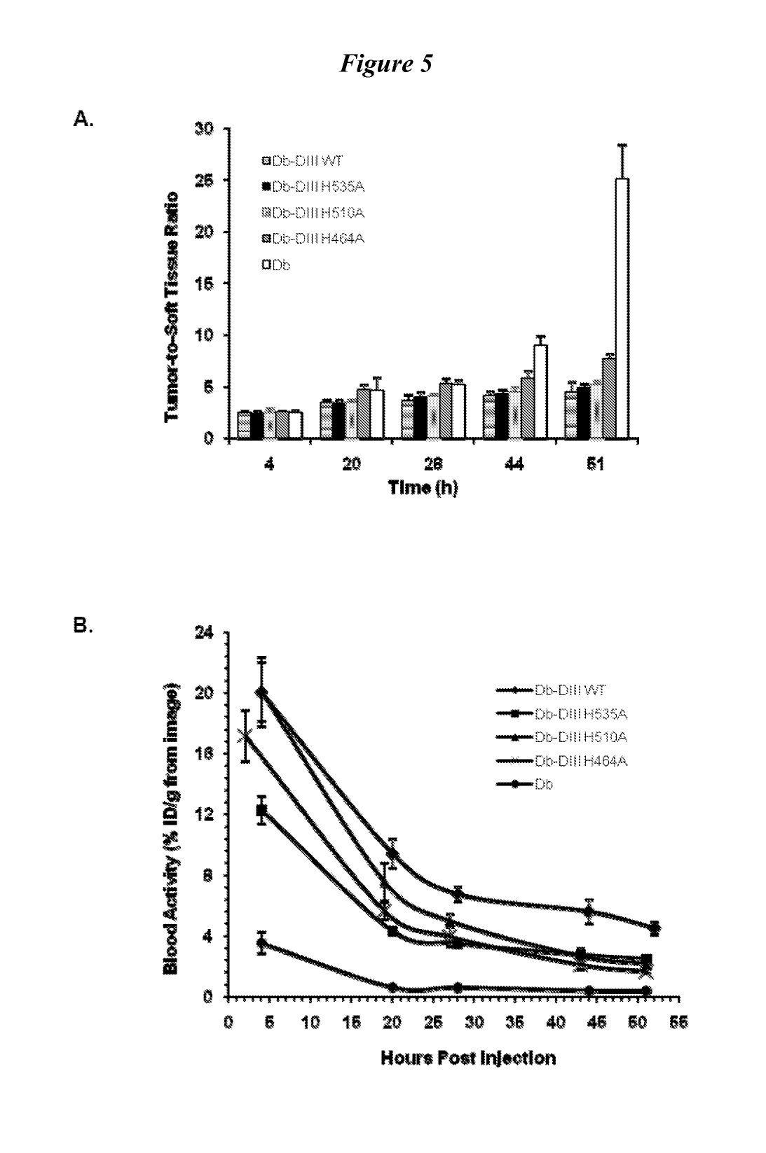

The ¹²⁴I labeling efficiency for the Db-DIII fusion proteins ranged from 63.9 to 81.5% and the injected specific activities were between 13.0 and 18.0 GBq/μmol. Serial small animal PET imaging allowed for comparison of the Db-DIII fusion proteins with the Db alone in vivo, in terms of tumor targeting and persistence in the circulation (FIG. 4). The images reveal that all five proteins target the LS 174T (CEA-positive) tumor. The tumor anatomical location is clearly seen on the CT image. Targeting was noted as early as 4 h for the Db and Db-DIII H464A, and 20 h for the Db-DIII WT, H535A and H510A molecules. The signal in the CEA-positive tumor persisted throughout the entire study (51 h) for all proteins, while the background (circulation activity) was variable. A statistical comparison of the T:ST ROI ratios of Db-DIII and Db proteins at different time points (FIG. 5A) showed that at 4 and 20 h all proteins exhibited T:ST ratios that were not significantly different from each other (P>0.05). From 28 h, through 44 h to 51 h, the Db T:ST ratio remained significantly larger than all of the Db-DIII proteins (P values ranging from 0.04 to 0.01). At 51 h H535A (P=0.03), H510A (P=0.02) and H464A (P=0.01) variants showed significantly higher T:ST ratios compared to the WT, due to a higher rate of soft tissue signal decline. However, the H510A T:ST ratio was not significantly different from the H535A (P=0.1). The H464A T:ST ratio was not significantly different from the H510A T:ST ratio either (P=0.07). The H464A T:ST ratio was significantly different from the H535A (P=0.02). PET image quantification of the radioactivity in blood (% ID/g) for each time point allowed for generation of blood activity curves (FIG. 5B) and calculation of the MRT for each protein in the blood (Table 1). Db-DIII WT exhibited significantly slower blood clearance kinetics compared to all histidine mutants and the Db alone (P<0.05). The Db-DIII H535A and H510A, as well as the H510A and H464A blood activity curves were not significantly different from each other (P=0.09 and P=0.08, respectively), while H535A was characterized by significantly longer blood persistence, compared to the H464A variant (P=0.01). Thus, the order from the longest to the shortest serum MRT is: Db-DIII WT>H535A>H510A>H464A>Db, where Db-DIII H535A has significantly longer circulation residence time compared to H464A. Biodistribution at 51 h confirmed the order of serum persistence (Table 2). The measured activity in blood for the Db-DIII proteins ranged from 4.0 to 1.6% ID/g, while the LS174T tumor uptake was between 2.5 and 1.3% ID/g, compared to 0.5% ID/g for the Db. Previous studies have shown that the radioiodinated T84.66 Db reaches maximum tumor uptake at 2 h after injection (13.68±1.49% ID/g), after which the activity in the tumor starts to decline (Wu et al., 1999). Tumor masses averaged 161 mg and 126 mg for LS174T and C6 tumors, respectively. It was noted that longer serum residence time was generally associated with higher LS174T tumor uptake. The CEA positive-to-negative tumor uptake ratios for the Db-DIII proteins ranged from 1.5 to 2.2, compared to 13 for the Db alone at 51 h.

TABLE 1
Biodistribution, showing the ¹²⁴I-labeleled Db-DIII proteins in descending order
(top to bottom), in terms of their persistence in the circulation (blood % ID/g).

| | Organ/Tissue (% ID/g) at 51 h (mean (SE)). | | | | | | | |
|---|---|---|---|---|---|---|---|---|
| | | | | | | LS174T | | |
| Protein | Blood | Liver | Spleen | Kidneys | Lungs | (+) | C6 (−) | Carcass |
| Db-DIII WT | 4.00 | 0.79 | 0.89 | 1.01 | 2.05 | 2.46 | 1.67 | 0.83 |
| | (0.22) | (0.03) | (0.11) | (0.11) | (0.20) | (0.19) | (0.30) | (0.12) |
| Db-DIII | 2.04 | 0.67 | 0.63 | 0.75 | 0.91 | 1.08 | 0.69 | 0.33 |
| H535A | (0.08) | (0.05) | (0.05) | (0.06) | (0.08) | (0.11) | (0.12) | (0.03) |
| Db-DIII | 1.77 | 0.54 | 0.58 | 0.53 | 0.98 | 1.10 | 0.72 | 0.40 |
| H510A | (0.14) | (0.06) | (0.03) | (0.08) | (0.07) | (0.07) | (0.14) | (0.02) |
| Db-DIII | 1.56 | 0.44 | 0.50 | 0.58 | 0.85 | 1.31 | 0.59 | 0.28 |
| H464A | (0.15) | (0.06) | (0.07) | (0.08) | (0.12) | (0.15) | (0.06) | (0.03) |
| Db | 0.08 | 0.19 | 0.10 | 0.33 | 0.10 | 0.52 | 0.04 | 0.03 |
| | (0.01) | (0.01) | (0.01) | (0.02) | (0.02) | (0.06) | (0.001) | (0.001) |

Note:
Groups of four mice per protein were analyzed. Organ uptake is expressed as % ID/g

TABLE 2
Estimated values of blood half-lives for the Db-DIII and Db in tumor
bearing athymic nude mice.

| Protein | Blood AUC¹ | First Mo² (h | MRT³ (h) |
|---|---|---|---|
| Db-DIII WT | 1449 | 178 | 56.7 |
| Db-DIII H535A | 470 | 66 | 25 |
| Db-DIII H510A | 528 | 38 | 20 |
| Db-DIII H464A | 354 | 24 | 17 |
| Db | 104 | 26 | 2.9 |

¹AUC is the area under the curve - ∫ u(t) dt from 0 to infinity.
²First Mo is the first moment: ∫t u(t) dt/∫ u(t) dt where u(t) is the measured (% ID/g) blood curve.
³Mean residence time is the same integral format except du/dt replaces u(t).

### Discussion

As a first step and a proof of principle that HSA DIII can act as a protein scaffold with tunable PK, we designed a fusion protein consisting of two components. One was the anti-CEA T84.66 Db, which is a small divalent antibody fragment that has been extensively studied in vivo. The anti-CEA Db exhibits a terminal β half life ranging from 2.89 h (¹²³I) to 3.04 h (²²²In) in LS174T (CEA-positive) tumor bearing mice (Yazaki et al., 2001). This Db has also been successfully fused to other proteins (i.e. Renilla or Gaussia Luciferases), where it retained its in vivo targeting capacity (Venisnik et al., 2007; Venisnik et al., 2006). Therefore, the Db makes a good model targeting molecule for a proof of concept study. The second component is the one that has unknown characteristics, namely the HSA DIII WT or one of its variants with mutated H535, H510 or H464 residue. The Db-DIII fusion proteins were expressed in mammalian cells to ensure proper folding. Expression levels were reasonable and affinity purification yielded proteins of molecular mass consistent with the calculated 101 kDa (FIG. 2A). The Db is a non-covalent dimer of two scFv molecules, which separate from each other under SDS-PAGE conditions and migrate around 25 kDa (Wu et al., 1999). We expected that the Db-DIII molecules would migrate as a scFv-DIII (˜48 kDa) species, as all cysteine residues, both in the DIII and scFv, are paired (Curry et al., 1998; Dugaiczyk et al., 1982; Wu, 1999). Interestingly, under non-reducing conditions the bulk of the protein remained in its dimeric form [(scFv-DIII)₂; 101 kDa], exhibiting increased structural stability under SDS conditions. After closer examination of the Db-DIII computer model (FIG. 3C), the linker length between the Db and DIII was reduced from 18 to 5 amino acids (data not shown). This alteration did not affect the migration pattern of the protein [(scFv-DIII)₂; FIG. 2A). Because of this unexpected behavior, the Db-DIII protein bands from the SDS-PAGE gel (FIG. 2A) were excised and the extracted protein was analyzed by mass spectrometry (data not shown). The results confirmed that the protein of approximately 101 kDa was indeed the Db-DIII. Elevated SDS and heat stability may be a result of polar, ionic interactions between the two scFv-DIII molecules, as is the case with β-glycosidase (Gentile et al., 2002). The molecular size of Db-DIII proteins was confirmed by size exclusion chromatography under physiologic conditions. The elution time of Db-DIII is close to another protein of similar molecular mass (scFv-Fc, 105 kDa), which elutes at approximately 27.3 min, under the same conditions (Kenanova et al., 2005). Being slightly smaller, the Db-DIII eluted at around 28.2 min, whereas the Db alone elutes at 38.2 min (Kenanova et al., 2005). In addition to high purity, the single peak on the chromatogram (FIG. 2C) revealed the integrity of the Db-DIII protein and that it exists as a single species. Analysis of the DIII-FcRn docking model (FIG. 3B) was useful in defining the possible FcRn interaction partners of H535, H510 and H464 residues and was also able to define their importance for FcRn binding. However, the actual ranking of the DIII histidine residues was determined by in vivo molecular imaging.

The strength of molecular imaging, specifically PET, is that the same individual can be imaged tomographically multiple times after injection of the tracer to extract quantitative information about PK, tumor targeting, cross-reactivity. In this study, mice bearing CEA-positive and negative xenografts were injected with ¹²⁴I-labeled Db-DIII or Db proteins and imaged at five different time points. This allowed for head to head comparison of the Db-DIII proteins with each other, as well as with the Db alone in terms of their persistence in the circulation and tumor targeting. Since the Db was the constant component, differences in PK among Db-DIII proteins were attributed to the function of the DIII. Thus, although indirectly, PET imaging enabled us to make conclusions about the behavior of the DIII protein in vivo.

Targeting agents with more rapid serum clearance achieve higher T:ST ratios at earlier time points. Therefore, solely based on the T:ST ROI ratios at each time point (FIG. 5A), we were able to deduce the order of blood clearance from fastest (highest T:ST ratio) to slowest (lowest T:ST ratio) as: Db>>Db-DIII H464A>H510A>H535A>WT. Interestingly, the statistical analysis showed that Db-DIII H510A was not significantly faster clearing than H535A. Both H535 and H510 residues are located in sub-domain DIIIb. This finding could suggest that within albumin the H510 and H535 residues may be redundant, playing a backup role for each other in case one is non-functional or not available for binding to the FcRn. This hypothesis remains to be elucidated. Simultaneous mutation of both residues can provide more insight. The same order of circulation clearance was also confirmed by the blood activity curves generated from quantifying the radioactivity in the mouse heart at each time point for every protein (FIG. 5B). Statistical comparison of the blood activity curves led to the same conclusion as above—Db-DIII H535A clears significantly slower than H464A, but not compared to H510A. Due to lack of more time points within the first 12 hours post tracer injection, calculation of MRT, rather than α and β half lives was more feasible. The MRT ranged from about 2.4 days for the Db-DIII WT to 17 h for the Db-DIII H464A, compared to 2.9 h for the Db alone. The overall size of the Db-DIII fusion proteins (101 kDa) is above the threshold for renal clearance (˜60 kDa). Therefore, Db-DIII proteins are eliminated through the hepatobiliary route, while the Db (55 kDa) is cleared through the kidneys. Thus, the difference in molecular mass between Db and Db-DIII proteins is largely responsible for the difference in MRT. However, the fact that the Db-DIII PK in vivo can be modulated through single amino acid mutation (same molecular mass) suggests that there is an additional molecular mechanism that governs serum PK in vivo apart from increase in molecular size (e.g. FcRn interaction). Furthermore, the mutations (H535A, H510A or H464A), allow for finer tuning of the overall protein serum residence time. One can choose from a spectrum of circulation half lives ranging from days to hours. This is advantageous when selecting for diagnostic or therapeutic agents with specific serum PK requirements.

The imaging studies clearly demonstrate the ability of the HSA DIII domain to increase the circulation persistence of the Db, while retaining tumor targeting. All Db-DIII proteins remained in blood significantly longer than the Db alone (P<0.05). Direct count of the radioactivity (% ID/g) remaining in blood for the mice injected with the Db-DIII fusion proteins was from 50 (WT) to 20 (H464A) fold higher than that for the Db injected mice at 51 h. Collectively, our findings suggest that the HSA DIII WT and mutants alone should be capable of tailoring the serum residence time of the moiety which they are attached to. The DIII ranking of blood clearance is expected to remain the same as the experimentally determined Db-DIII order. The DIII WT was also able to prolong the serum persistence of the Db slightly more than the entire HSA molecule did to the T84.66 scFv (Yazaki et al., 2008). At 48 h post injection of ¹²⁵I-labeled anti-CEA scFv-HSA fusion protein (˜90 kDa) in LS174T xenografted athymic nude mice, the remaining activity in blood was 2.79% ID/g, compared to 4.00% ID/g for the ¹²⁴I-labeled Db-DIII WT at 51 h (Table 2). This difference can possibly be explained by the larger molecular mass of Db-DIII. Nevertheless, it suggests that DIII is both necessary and sufficient for maintaining the serum half life of the entire HSA molecule. H464 (located in DIIIa) appears to have the biggest effect on FcRn binding and circulation persistence. Additionally, since H535A and H510A mutations produce significantly faster blood clearances compared to the WT, we can conclude that both subdomains DIIIa and DIIIb participate in maintaining serum persistence.

The purpose of the Db-DIII proteins was to elucidate the potential of the HSA DIII for use as a single domain scaffold with controlled PK. Expression of the DIII WT and variants without a targeting moiety, and evaluation of their PK in vivo is the next step towards selection of DIII scaffolds, exhibiting properties optimal for imaging or therapy applications. The DIII scaffolds described in this work may be used for grafting or chemically conjugating tumor targeting molecules (peptides, aptamers, small chemical molecules) or directly for creating display combinatorial libraries. The targeted scaffolds with suitable PK for imaging may be used for diagnostic purposes. Alternatively, potential anti-tumor drugs could be conjugated to the targeted scaffolds with optimal characteristics for cancer treatment.

### Example 2

### Binding Studies with Alexa Fluor 647 Conjugated DIII Proteins

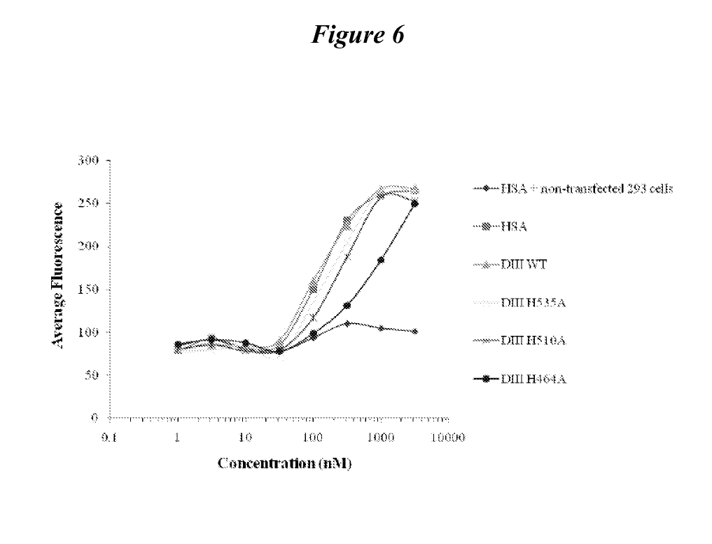

The fluorophore Alexa Fluor 647 (1.25 kDa) was conjugated to HSA, DIII WT, H535A, H510A and H464A proteins using the Alexa Fluor 647 Protein Labeling Kit (Invitrogen, Eugene, Oreg.) according to manufacturer's instructions. Dilutions of each fluorescent protein ranging from 0.316 to 3160 nM (in triplicates) were incubated with confluent 293 human embryonic kidney cells expressing human FcRn (Petkova et al., Int Immunol. 2006; 18:1759-1769) at pH 6.5 in a round bottom 96-well plate. Dilutions of Alexa Fluor 647 conjugated HSA were also incubated with 293 cells devoid of FcRn expression (control reaction). Following a washing step with 1×PBS (pH 6.5), the cells were imaged by the Maestro™ In-Vivo Fluorescence Imaging System (CRi, Woburn, Mass.) using Deep Red (671-705 nm) excitation and Red (700 nm longpass) emission filters. Same size regions of interest (ROI) were drawn in each well and the fluorescent signal was measured and averaged for each dilution. A binding curve was generated with mean fluorescence as a function of Alexa-Fluor 647 conjugated DIII protein concentration. The DIII concentration at which 50% fluorescence was measured signified the DIII protein relative binding affinity for FcRn (see, FIG. 6).

### Binding of DIII Proteins to Human FcRn

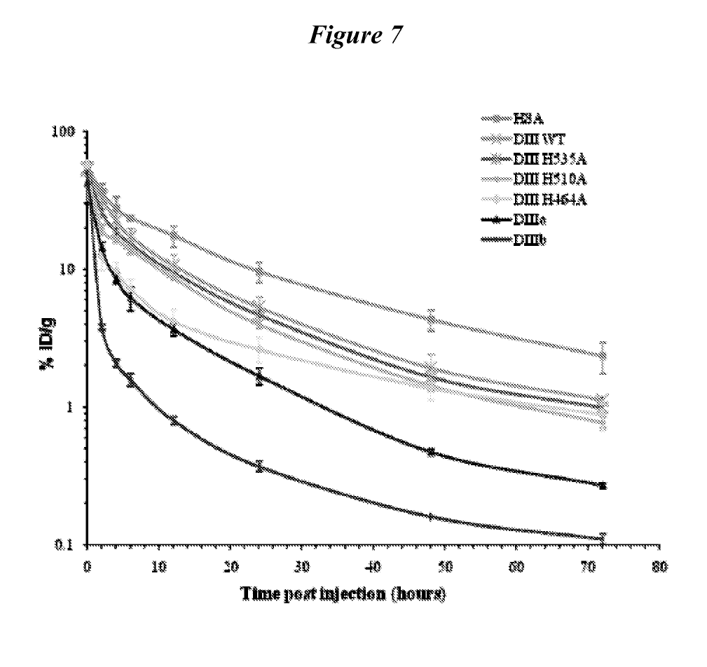

FIG. 7 depicts the binding curves of fluorophore conjugated HSA and DIII proteins. The more left shifted curves (HSA and DIII WT) represent stronger binding to FcRn expressing 293 cells with relative binding affinity in the range of 100 nM, followed by DIII H535A and H510A (˜200 and 300 nM, respectively) and DIII H464A with lowest relative binding affinity of about 1 μM. Alexa Fluor 647 conjugated HSA did not bind 293 cells (devoid of FcRn), thus suggesting specific interaction with FcRn. Based on the cell binding studies, the order of binding affinity to human FcRn from high to low is as follows: HSA>DIII WT>DIII H535A>DIII H510A>DIII H464A.

### Circulation Half-Lives of DIII Proteins in Mice

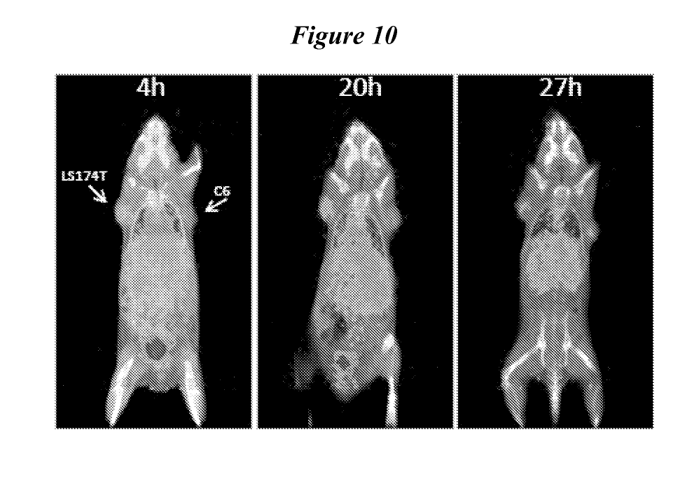

The ¹³¹I labeling efficiency for the HSA and DIII proteins ranged from 39.6 to 93.6% and the injected specific activities were between 1.5 and 3.1 μCi/μg. The blood activity curves of intact HSA and all DIII proteins (FIG. 10) show the same order of elimination as the one observed with Db-DIII fusion proteins, with the addition of DIIIa and DIM. Furthermore, the decrease in relative binding affinity of HSA and DIII proteins for FcRn (FIG. 7) is proportional to the decrease in circulation persistence. Table 3 summarizes the estimated values of blood half-lives. The order of blood clearance, starting from slow to fast is as follows: HSA>DIII WT>DIII H535A>DIII H510A>DIII H464A>DIIIa>DIIIb. The slow phase (β) half life span from the slowest (DIII WT) to the fastest clearing (DIM)) protein is about 2 fold, with t_{1/2}β ranging from 15.3 to 6.9 h. This spectrum of circulation residence times allows for one to choose the DIII platform that can fit best the desired application (e.g. therapy, imaging).

TABLE 3
Estimated values of blood half-lives for the HSA and DIII proteins
in Balb/c mice.

| | | | | | t_{1/2}α | t_{1/2}β | |
|---|---|---|---|---|---|---|---|
| Agent | Aα¹ | Aβ | k1 | k2 | (h)² | (h) | AUC³ |
| HSA | 26.88 | 27.49 | 0.454 | 0.04 | 1.52 | 17.3 | 746 |
| DIII WT | 36.2 | 16.81 | 0.3479 | 0.04534 | 1.99 | 15.29 | 474.8 |
| DIII H535A | 32.83 | 21.98 | 0.7871 | 0.06459 | 0.881 | 10.73 | 382 |
| DIII H510A | 30.74 | 21.41 | 1.798 | 0.07108 | 0.386 | 9.75 | 318 |
| DIII H464A | 44.95 | 11.41 | 1.438 | 0.068 | 0.482 | 10.2 | 199 |
| DIIIa | 33.30 | 9.907 | 0.874 | 0.07763 | 0.79 | 8.93 | 165.7 |
| DIIIb | 49.22 | 2.967 | 1.788 | 0.1009 | 0.388 | 6.87 | 56.7 |

¹Amplitudes of the two components are given by Aα and Aβ, where the sum of Aα and Aβ is the total % ID/g.
²t_{1/2}α = ln2/k1 and t_{1/2}β = ln2/k2.
³Area under the curve (AUC) is a time integral of the blood uptake (% ID/g × h).

### Example 3

Generation and conjugation of aptamer molecules to selected DIII scaffold(s). Modified target specific aptamer, containing nuclease-resistant pyrimidines 2′-Fluoro UTP and 2′F CTP can be generated by runoff transcription from double-stranded DNA template bearing a T7 RNA polymerase promoter. The transcription reaction can be carried out using the Y639F mutant T7 RNA polymerase. The nucleotides used in the reaction will consist of ATP, GTP, 2′F dCTP and 2′F dUTP. For conjugation of the aptamer to the DIII scaffold, succinimidyl 6-hydrazinonicotinamide acetone hydrazone (SANH) can be reacted with the DIII scaffold lysine residues (Figure below). The bis-aryl hydrazone bond between the two molecules is UV traceable at 354 nM, therefore the conjugation ratio can be determined spectroscopically. Following purification, all conjugated products can be evaluated for their ability to bind the target in vitro (cells) and then in vivo (xenografted mice).

Conjugation chemistry of the aptamer to the scaffold DIII (shown in filled circle). A desalting step is necessary after the first reaction step to remove the non-reacted SANH.

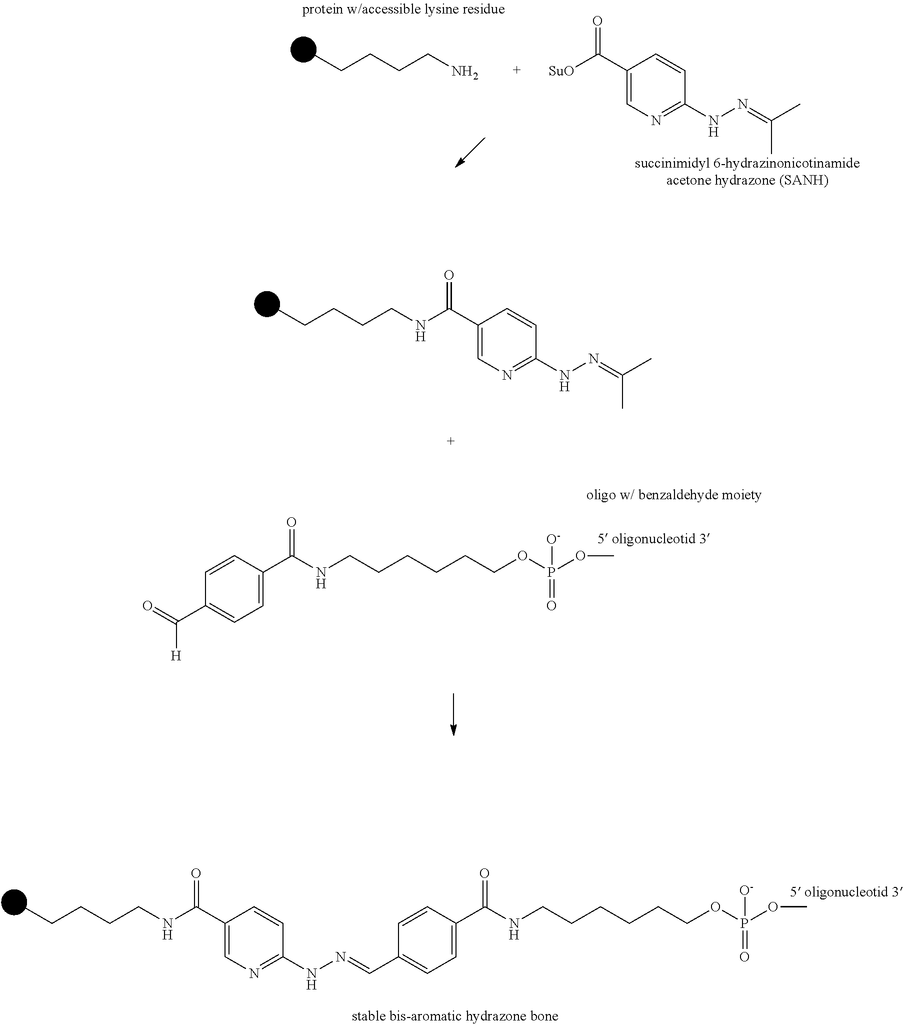

The bioconjugation of target specific peptides or other proteins is accomplished through the use of two heterobifunctional linkers. One is an aromatic hydrazine[6-hydrazinonicotinamide (HyNic)] and is synthesized either at the C- or N-terminus of the peptide or protein. The other is an aromatic aldehyde[4-formylbenzamide (4FB)] attached to random lysine (K) residues on the DIII protein. The 4FB incorporation process is referred to as “modification” of DIII. Once modified, functionalized DIII and peptide molecules are desalted to remove excess linker and to exchange the biomolecules int a conjugation-compatible buffer system. The two modified biomolecules are then mixed together and conjugation occurs through the formation of a bis-aryl hydrazone bond between the two species that is thermally stable and also can be measured spectroscopically at A₃₅₄ₙₘ. The peptide/DII ratio is then calculated. Commercially available reagents from SoluLink (San Diego, Calif.) can be used to complete this conjugation reaction.

### Example 4

### Evaluation of Anti-CEA Peptide-DIIIb Conjugate

CEA specific, cyclic peptide (SDWVCEFIKSQWFCNVLASG, Kd=160 nM) (SEQ ID NO: 7) was commercially synthesized with a HyNic group at the C terminus (SoluLink). When dissolved in aqueous solution, the peptide precipitated. Lack of solubility in water immediately renders this peptide inappropriate for in vivo application. Prior to conjugation, the HyNic modified peptide was dissolved in dimethyl formamide (DMF) organic solvent. The purified DIIIb was modified to incorporate the 4FB moiety at random lysine residues following protocols provided by SoluLink. After conjugation, the peptide/DIII ratio was determined by measuring A₃₅₄ₙₘ to be an average of 2 CEA specific peptides for every DIIIb molecule and the conjugate was soluble in aqueous solutions. Size exclusion chromatography using Superdex 200 column (GE Healthcare Piscataway, N.J.) was used for purification. Purified anti-CEA peptide-DIIIb conjugates were then radiolabeled with ¹²⁴I and injected intravenously in four athymic nude mice bearing LS174T (CEA positive) and C6 (CEA negative) tumors. Mice were imaged by small animal PET/CT at 4, 20 and 27 h, after which mice were euthanized, dissected and tissues/organs were counted in a gamma well counter. Table 4 below shows the calculated percent injected dose per gram (% ID/g) at 27 h post injection.

The PET/CT images (FIG. 10) demonstrate the ability of the peptide-DIIIb conjugate to target the CEA positive tumor. High circulation activity is noted, suggesting that the DIIIb function to prolong the circulation half life of the tumor targeting peptide is maintained. However, the targeting moiety (peptide) is not capable of binding the target efficiently, leading to dissociation of the peptide-DIIIb conjugate and getting it back in the circulation. This is confirmed by the biodistribution data (Table 4), with relatively low LS174T tumor uptake and high blood activity at 27 h post injection.

TABLE 4
Biodistribution of ¹²⁴I-labeled anti-CEA peptide-DIIIb conjugate in tumor
bearing mice (n = 4)

| Tissue/Organ | % ID/g | Std. Deviation |
|---|---|---|
| Blood | 3.00 | 0.32 |
| Liver | 1.42 | 0.08 |
| Spleen | 1.04 | 0.16 |
| Kidneys | 3.66 | 0.66 |
| Lungs | 2.36 | 0.87 |
| Muscle | 0.30 | 0.03 |
| Stomach | 1.07 | 0.19 |
| LS174T Tumor | 1.53 | 0.20 |
| C6 Tumor | 1.26 | 0.20 |
| Carcass | 0.56 | 0.08 |

Tumor/muscle ratio of 5.1 is acceptable and comparable to antibody imaging. The tumor/blood and (CEA positive)tumor/(CEA negative)tumor ratios are relatively low (0.51 and 1.2, respectively), indicative of high activity in blood and diminished tumor targeting. This observation once again suggests that the conjugate remains in blood sufficiently long (DIIIb function) but the peptide is not proficient in binding the target (CEA expressed by LS174T tumors). We conclude that peptides with higher affinity (e.g., Kd<10 nM) are preferred for imaging for conjugation to DIII as avidity alone cannot make up for poor affinity.

### REFERENCES

Aina, O. H., Liu,R., Sutcliffe, J. L., Marik, J., Pan, C. X. and Lam, K. S. (2007) Mol. Pharm., 4, 631-651.
Andersen, J. T., Dee Qian, J. and Sandlie, I. (2006) Eur. J. Immunol., 36, 3044-3051. Barbas, A. S. and White, R. R. (2009) Curr. Opin. Investig. Drugs, 10, 572-578.
Bebbington, C. R., Renner, G., Thomson, S., King, D., Abrams, D. and Yarranton, G. T. (1992) Biotechnology (N Y), 10, 169-175.
Bembenek, S. D., Tounge, B. A. and Reynolds, C. H. (2009) Drug Discov. Today, 14, 278-283.
Bos, O. J., Labro, J. F., Fischer, M. J., Wilting, J. and Janssen, L. H. (1989) J. Biol. Chem., 264, 953-959.
Burger, A. M., Hartung, G., Stehle, G., Sinn, H. and Fiebig, H. H. (2001) Int. J. Cancer, 92, 718-724.
Carmichael, J. A., Power, B. E., Garrett, T. P., Yazaki, P. J., Shively, J. E., Raubischek, A. A., Wu, A. M. and Hudson, P. J. (2003) J. Mol. Biol., 326, 341-351.
Chaudhury, C., Brooks, C. L., Carter, D. C., Robinson, J. M. and Anderson, C. L. (2006) Biochemistry, 45, 4983-4990.
Chaudhury, C., Mehnaz, S., Robinson, J. M., Hayton, W. L., Pearl, D. K., Roopenian, D. C. and Anderson, C. L. (2003) J. Exp. Med., 197, 315-322.
Chen, R., Li, L. and Weng, Z. (2003) Proteins, 52, 80-87. Curry, S, Mandelkow, H., Brick, P. and Franks, N. (1998) Nat. Struct. Biol., 5, 827-835.
D'Argenio, D. Z. and Schumitzky, A. (1979) Comput. Programs Biomed., 9, 115-134.
Defrise, M., Kinahan, P. E., Townsend, D. W., Michel, C., Sibomana, M. and Newport, D. F. (1997) IEEE Trans Med. Imaging, 16, 145-158.
Dugaiczyk, A., Law, S. W. and Dennison, O. E. (1982) Proc. Natl. Acad. Sci. USA, 79, 71-75.
Friedman, M., Nordberg, E., Hoiden-Guthenberg, I., Brismar,H., Adams, G. P., Nilsson, F. Y., Carlsson, J. and Stahl, S. (2007) Protein Eng. Des. Sel., 20, 189-199.
Gentile, F., Amodeo, P., Febbraio, F., Picaro, F., Motta, A., Formisano, S, and Nucci, R. (2002) J. Biol. Chem., 277, 44050-44060.
Gronwall, C. and Stahl, S. (2009) J. Biotechnol., 140, 254-269.
Kenanova,V., et al. (2005) Cancer Res., 65, 622-631.
Kratz, F., Muller-Driver, R., Hofmann, I., Drevs, J. and Unger, C. (2000) J. Med. Chem., 43, 1253-1256.
Loening, A. M. and Gambhir, S. S. (2003) Mol. Imaging, 2, 131-137.
Martin, W. L., West, A. P., Jr., Gan, L. and Bjorkman, P. J. (2001) Mol. Cell, 7, 867-877.
Melder, R. J., et al. (2005) Cancer Immunol. Immunother., 54, 535-547.
Muller, D., Karle, A., Meissburger, B., Hofig, I., Stork, R. and Kontermann, R. E. (2007) J. Biol. Chem., 282, 12650-12660.
Nuttall, S. D. and Walsh, R. B. (2008) Curr. Opin. Pharmacol., 8, 609-615.
Olafsen, T., Kenanova, V. E. and Wu, A. M. (2006) Nat. Protoc., 1, 2048-2060.
Osborn, B. L., Olsen, H. S., Nardelli, B., Murray, J. H., Zhou, J. X., Garcia, A., Moody, G., Zaritskaya, L. S, and Sung, C. (2002) J. Pharmacol. Exp. Ther., 303, 540-548.
Saerens, D., Ghassabeh, G. H. and Muyldermans, S. (2008) Curr. Opin. Pharmacol., 8, 600-608.
Simonsen, S. M., Sando, L., Rosengren, K. J., Wang, C. K., Colgrave, M. L., Daly, N. L. and Craik, D. J. (2008) J. Biol. Chem., 283, 9805-9813.
Sugio, S., Kashima, A., Mochizuki, S., Noda, M. and Kobayashi, K. (1999) Protein Eng., 12, 439-446.
Venisnik, K. M., Olafsen, T., Gambhir, S. S. and Wu, A. M. (2007) Mol. Imaging. Biol., 9, 267-277.
Venisnik, K. M., Olafsen, T., Loening, A. M., Iyer, M., Gambhir, S. S. and Wu, A. M. (2006) Protein Eng. Des. Sel., 19, 453-460.
Wosikowski, K., Biedermann, E., Rattel, B., Breiter, N., Jank, P., Loser, R., Jansen, G. and Peters, G. J. (2003) Clin. Cancer Res., 9, 1917-1926.
Wu, A. M., Williams, L. E., Zieran, L., Padma, A., Sherman, M., Bebb, G. G., Odom-Maryon, T., Wong, J. Y. C., Shively, J. E. and Raubitschek, A. A. (1999) Tumor Targeting, 4, 47-58.
Wu, A. M., Williams, L. E., Zieran, L., Padma, Q., Sherman, M. A., Bebb, G. G., Odom-Maryon, T., Wong, J. Y. C., Shively, J. E., and Raubitschek, A. A. (1999) Tumor Targeting, 4.
Yazaki, P. J., Kassa, T., Cheung, C. W., Crow, D. M., Sherman, M. A., Bading, J. R., Anderson, A. L., Colcher, D. and Raubitschek, A. (2008) Nucl. Med. Biol., 35, 151-158.
Yazaki, P. J., Wu, A. M., Tsai, S. W., Williams, L. E., Ikler, D. N., Wong, J. Y., Shively, J. E. and Raubitschek, A. A. (2001) Bioconjug. Chem., 12, 220-228.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A construct comprising:
a) a protein scaffold, wherein the scaffold comprises Domain III, Domain IIIa, or Domain IIIb of human serum albumin or a polypeptide having substantial sequence identity to the Domain III, the Domain IIIa, or the Domain IIIb;
b) a targeting moiety in covalent linkage to the protein scaffold; and
c) a therapeutic moiety or an imaging moiety in covalent linkage to the protein scaffold.

2. The construct of claim 1, wherein the targeting moiety is a ligand which binds a receptor of a target tissue or cell.

3. The construct of claim 1, wherein the targeting moiety is an antibody, or an immunologically active fragment thereof, which binds a tumor specific antigen.

4. The construct of claim 3, wherein the antibody is an immunologically active fragment of the antibody, a diabody, a triabody, or a minibody.

5. The construct of claim 1, wherein the targeting moiety is an aptamer.

6. The construct of claim 5, wherein the aptamer binds a tumor specific antigen.

7. The construct of claim 2, wherein the ligand binds to a protein overexpressed in a target tissue or cell.

8. The construct of claim 2, wherein the target tissue or cell is a cancer.

9. The construct of claim 1, wherein at least one of the targeting moiety, imaging moiety, or therapeutic moiety is covalently attached to the scaffold by a non-peptide linker.

10. The construct of claim 1, wherein the substantial identity is 90%.

11. The construct of claim 1, wherein the substantial identity is 95%.

12. The construct of claim 1, wherein at least one of the targeting moiety, imaging moiety, or therapeutic moiety is covalently attached to the scaffold by a heterobifunctional cross linker, a homobifunctional crosslinker, a zero-length cross linker, a disulfide bond, or a physiologically cleavable cross-linker.

13. The construct of claim 1, wherein the targeting moiety is covalently attached to the scaffold by a linker which is from 2 to 20 atoms in length.

14. The construct of claim 1, wherein imaging moiety or the therapeutic moiety are attached to the scaffold by a linker which is from 2 to 20 atoms in length.

15. The construct of claim 1, wherein the construct has a molecule weight of less than 40 kda.

16. The construct of claim 1, wherein the construct has a molecular weight of less than 30 kda.

17. The construct of claim 1, wherein the construct has a molecular weight of less than 20 kda.

18. The construct of claim 1, wherein the Domain III is wildtype or has a mutation at H535, H510, or H464.

19. The construct of claim 18, wherein the mutation is H535A, H510A, or H464A.

20. The construct of claim 1, wherein the protein scaffold consists essentially of the Domain III, Domain IIIa, or Domain IIIb.

21. The construct of claim 1, with the proviso that the targeting moiety is not connected to the scaffold by a peptide bond.

22. The construct of claim 1, with the proviso that the imaging and therapeutic moieties are not connected to the scaffold by a peptide bond.

23. The construct of claim 1, wherein the construct comprises the therapeutic agent.

24. The construct of claim 23, wherein the therapeutic moiety is a drug.

25. The construct of claim 1, wherein the therapeutic moiety is a therapeutic radionucleide, a cytotoxic drug, a cytokine, a chemotherapeutic agent, a radiosensitizing agent, or an enzyme.

26. The construct of claim 25, wherein a plurality of the therapeutic moiety are covalently linked to the scaffold.

27. The construct of claim 1, wherein the construct comprises the imaging agent.

28. The construct of claim 27, wherein the imaging agent is selected from the group consisting of radionuclides, diamagnetic materials, fluorescent markers, chromogens, quantum dots, nanoparticles, and bioluminescent enzymes.

29. The construct of claim 27, wherein a plurality of the imaging agent are covalently linked to the scaffold.

30. A method of detecting a biomolecule associated with a disease or condition in a subject, comprising administering to a subject suspected of having, or having, the disease or condition a construct of claim 27, wherein the targeting moiety of the construct binds the biomolecule and the imaging agent of the construct is detected.

31. The method of claim 30, wherein the presence of absence of the disease or condition is diagnosed.

32. The method of claim 30, wherein the biomolecule is a tumor specific antigen and the disease or condition is cancer.

33. The method of claim 30, wherein the biomolecule is a cell surface receptor or protein which is overexpressed or underexpressed in the cells of a subject having the condition.

34. A method of treating a disease or condition associated with the presence of overexpression of a biomolecule in a tissue, said method comprising administering to a subject having the disease or condition a therapeutically effective amount of the construct of claim 23, wherein the targeting moiety of the construct binds the biomolecule and the therapeutic agent treats the disease or condition.

35. The method of claim 34, wherein the targeting moiety binds a tumor specific antigen of a cancer and the disease or condition is the cancer, and the therapeutic agent is a therapeutic radionucleide, a cytotoxic drug, a cytokine, or a chemotherapeutic agent.

36. A pharmaceutical or diagnostic composition comprising a construct of claim 1 and a physiologically acceptable excipient or carrier.

37. Use of a construct of claim 1, in the manufacture of a medicament for treating a disease or condition.

38. Use of a construct of claim 1, in the manufacture of a diagnostic for detecting a disease or condition.
